(19) United States
(12) Patent Application Publication
(10) Pub. No.: US 2022/0333177 A1
(43) Pub. Date: Oct. 20, 2022

(54) DIGITAL BIOMOLECULES DETECTION AND/OR QUANTIFICATION USING ISOTHERMAL AMPLIFICATION

(71) Applicants: CENTRE NATIONAL DE LA RECHERCHE SCIENTIFIQUE (CNRS), Paris (FR); INSTITUT NATIONAL DE LA SANTE ET DE LA RECHERCHE MEDICALE (INSERM), Paris (FR); ECOLE SUPERIEURE DE PHYSIQUE ET DE CHIMIE INDUSTRIELLES DE LA VILLE DE PARIS, Paris (FR); PARIS SCIENCES ET LETTRES, Paris (FR); UNIVERSITE DE PARIS, Paris (FR)

(72) Inventors: Yannick RONDELEZ, PARIS (FR); Guillaume GINES, PARIS (FR); Roberta LIMA DE CASTRO MENEZES, PARIS (FR); Valérie TALY, BOURG LA REINE (FR)

(73) Assignees: CENTRE NATIONAL DE LA RECHERCHE SCIENTIFIQUE (CNRS), Paris (FR); INSTITUT NATIONAL DE LA SANTE ET DE LA RECHERCHE MEDICALE (INSERM), Paris (FR); ECOLE SUPERIEURE DE PHYSIQUE ET DE CHIMIE INDUSTRIELLES DE LA VILLE DE PARIS, Paris (FR); PARIS SCIENCES ET LETTRES, Paris (FR); UNIVERSITE DE PARIS, Paris (FR)

(21) Appl. No.: 17/613,990

(22) PCT Filed: May 27, 2020
(86) PCT No.: PCT/EP2020/064771

§ 371 Date: Nov. 24, 2021

(30) Foreign Application Priority Data
May 27, 2019 (EP) 19305669.4

(51) Int. Cl. C12Q 1/6848; C12Q 1/6886

(52) U.S. Cl. CPC C12Q 1/6848; C12Q 1/6886; C12Q 2600/112

(57) ABSTRACT

The present invention relates to a digital method for detecting and/or quantifying at least one target biomolecules in a sample, said biomolecules being selected from DNA, RNA, and proteins based on isothermal amplification. The present invention further relates to different applications of the digital method and to a kit.

### FIELD OF THE INVENTION

The present invention relates to a digital method for detecting and/or quantifying biomolecules such as DNA, RNA and proteins in a sample, based on isothermal amplification having specific molecular design. The present invention further relates to a method for diagnosis of diseases selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases and to a method for agro diagnosis. It also relates to methods for detections of biomarkers (biomolecules) in food agri-food field and in the environment. All of these diagnosis and detection methods comprise the use of the digital method of the invention. The present invention also relates to a kit for detecting and/or quantifying at least one target biomolecule, said kit comprising enzymes, oligonucleotides and partitioning agents.

### BACKGROUND OF THE INVENTION

Several biomolecules such DNA, RNA or proteins are used as attractive diagnostic, prognostic or predictive biomarkers. These molecules and particularly the nucleic acids have accumulated clinical evidences that they are closely related to various diseases (cancers, neuronal or cardiovascular diseases, diabetes, etc.). Moreover, they are present in the bodily fluids and thus accessible via minimally invasive liquid biopsies (serum, plasma, urine). Circulating biomarkers can be assessed repeatedly enabling a regular follow up of treatments and relapses, large scale population screening or early diagnostic.

The detection of these biomolecules is often a challenging task since the obtained results have to be accurate in order to be used for clinical purpose. Hence, the precise measurement of such biomarkers is the critical bottleneck, which encourages the development of sensitive, specific and quantitative detection technologies.

The most used technique for sensitive detection of proteins is the enzyme-linked immunosorbent assay (ELISA) and of nucleic acids, the polymerase chain reaction (PCR), for detecting DNA and the reverse transcription-quantitative polymerase chain reaction (RT-qPCR) for detecting RNA. All these techniques allow specific and sensitive detection but have some limitations and need to be enhanced.

For example, currently the detection of DNA by PCR and related methods requires the amplification of the target nucleic acid. This may be prone to nonspecific or poor signal amplification that decrease the specificity and the sensitivity of the detection method.

With respect to RNA detection, despite the high sensitivity of RT-qPCR, the technique possesses some drawbacks: the RT step is known to introduce significant bias in the quantification (Bustin et al., 2015); primers and probe must be designed for each target and rely on sophisticated design due to the short length of the target; the thermocycling protocol should be optimized for each assay; the target itself is amplified and is therefore a hazardous source of carry-over contamination (Aslanzadeh et al., 2004); PCR reaction is known to be inhibited in biological samples (Opel et al. 2010). Additionally, the procedure relies on a real-time tracking of the amplification and requires standard calibration, thus giving only access to a qualitative estimate of the amount of biomolecule.

The above-mentioned drawbacks can be partially overcome by using a digital procedure based on the isolation and analysis of single nucleic acid molecules in small compartments.

Digital techniques have several advantages, in particular: i) they are compatible with endpoint assays and do not require continuous monitoring of the reaction; ii) they provide absolute quantification without calibration standards; iii) they provide ultimate sensitivity; iv) they also improve the sensitivity and specificity of the assay especially in the case of rare targets within complex background.

However, an assay can be transferred to the digital format only if its bulk sensitivity is already higher than the concentration corresponding to a single target per compartment. For example, if the compartments that are used for the digital procedure have an internal volume of around one nanoliter, the concentration of a single molecule in such a compartment is around the femtomolar and an amplification technique with a limit of detection lower than the femtomolar will be required for the digital assay. An amplification method with a limit of detection higher than the femtomolar will not be usable in this context. Although PCR generally fulfil the requisite of high sensitivity and provides a powerful method for precise and absolute quantification of biomolecules, particularly nucleic acids, it retains the weaknesses of RT-qPCR for assessment in digital format (Campomenosi et al., 2016).

Isothermal alternatives (Zhao et al., 2015) have been proposed (EXPAR, LAMP, RCA, HCR etc.) that rely on simpler one-step protocols, do not require thermocycling and free themselves from the reverse transcription reaction. Despite a competing sensitivity, these techniques suffer from nonspecific amplification reaction that make them ill-adapted to a digital readout. For example, Zhang et al. (Zhang et al., 2015) clearly demonstrated these limitations for EXPAR (Exponential Amplification Reaction), a method very well known for being prone to fast nonspecific amplification. When adapted to a digital format, the authors showed that empty droplets amplify only a few minutes after target-containing droplets, thus demonstrating that isothermal nucleic acid amplification method, while showing good sensitivity in bulk, cannot be transposed to a robust digital format.

Robust methods for digital measurement of enzymes and nucleic acids, particularly of microRNA are therefore not currently available.

The purpose of the present invention is to provide a method allowing absolute quantification and higher sensitivity when detecting biomolecules used as biomarkers, such as biopolymers, particularly nucleic acids and enzymes, which method is based on the digitalization of a specific isothermal amplification method but without the above cited drawbacks. Moreover, the present invention aims to provide such method which may be easily performed in one system.

### SUMMARY OF THE INVENTION

The inventors of the present invention have previously developed a method for eliminating background amplification for the detection of nucleic acids (WO2017140815). They surprisingly found that this analog method may be successfully digitalized in order to obtain a digital method allowing to increase the sensitivity of a detection of biomolecules used as biomarkers, to quantify accurately these one in bulk measurement without necessity of continuous monitoring of the amplification reaction, or using calibration curves.

According to the first aspect, the present invention thus relates to a digital method for detecting and/or quantifying at least one biomolecule in a sample, comprising the following steps:
a) mixing said sample with a mixture including a buffer, enzymes, a first oligonucleotide which is an amplification oligonucleotide, a second oligonucleotide which is a leak absorption oligonucleotide and a third oligonucleotide which is a target-specific conversion oligonucleotide;
b) partitioning the mixture obtained in step a) into several compartments so that a fraction of the compartments does not contain the target biomolecule;
c) converting the target biomolecule into a signal, said signal being preferably a DNA single strand
d) amplifying the signal, and
e) detecting and/or measuring said amplified signal in each compartment.

The main advantages of this method are that it allows rapid and selective detection of biomolecules such as DNA, RNA and proteins and also an absolute quantification of these ones.

Particularly when detecting and/or quantifying RNA, compared to the RT-PCR analog method, the advantage of the method of the invention is 1) the reverse transcription step, which may hamper the quantitativity of assay is not needed; 2) the high temperature and temperature cycling required for the PCR steps are not necessary; 3) the isothermal amplification is robust to many chemicals which may be found in crude samples and has been shown to inhibit the PCR reactions (Huggett et al. 2008) the cross-contamination issues are avoided because the target biomolecule is not amplified.

The inventors also demonstrated that the method of the present invention may be used to detect biomolecules directly from any type of sample containing biomolecules, for example a blood sample and other biological fluids.

The specificity, the simplicity and the rapidity of this method allow to use it in medical diagnosis methods, particularly for diagnosis of diseases such as cancer, neuronal diseases, cardiovascular diseases, inflammatory diseases, autoimmune diseases, diseases due to a viral or bacterial infection, skin diseases, skeletal muscle diseases, dental diseases and prenatal diseases.

According to the second aspect, the present invention also relates to an in vitro method for diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases and prenatal diseases, said diagnostic method comprising the use of the digital method.

The specificity, the sensitivity, the simplicity and the rapidity of the digital method of the invention allow to use it in agro diagnosis methods particularly for diagnosis of diseases caused by biotic stress such as infectious and parasitic diseases, or caused by abiotic stress such as nutritional deficiencies or unfavorable environments.

According to the third aspect, the present invention also relates to an in vitro method for agro diagnosis of a disease selected from the group comprising:
diseases caused by biotic stress, preferably by infectious and/or parasitic origin, or
diseases caused by abiotic stress, preferably caused by nutritional deficiencies and/or unfavorable environment,
   said method comprising the use of the digital method of the invention. For implementing the methods of the present invention, it is also provided a kit.

According to fourth aspect, the present invention thus relates to a kit for detecting and/or quantifying at least one target biomolecule comprising:
a) a mixture of enzymes, preferably selected from the group comprising polymerase, nicking enzyme or restriction enzymes and exonuclease;
b) a mixture of oligonucleotides comprising a first oligonucleotide which is an amplification oligonucleotide, a second oligonucleotide which is a leak absorption oligonucleotide and a third oligonucleotide which is a target-specific conversion oligonucleotide and optionally a fourth oligonucleotide which is a reporting probe and
c) partitioning agent, preferably a water-in-oil emulsion.

### DETAILED DESCRIPTION

As indicated above, the present invention concerns the adaptation of recent background-free amplification chemistry to a digital readout, thus providing absolute quantification of biomolecule targets.

Particularly, the adapted background-free amplification chemistry which comprises eliminating background amplification in isothermal amplification of biomolecules targets, particularly of nucleic acid targets, is those implemented by some of the inventors of the present invention and which is disclosed in the international application WO2017140815.

According to the first aspect, the present invention thus relates to a digital method for detecting and/or quantifying at least one biomolecule in a sample comprising the following steps:
a) mixing said sample with a mixture including a buffer, enzymes, a first oligonucleotide which is an amplification oligonucleotide, a second oligonucleotide which is a leak absorption oligonucleotide and a third oligonucleotide which is a target-specific conversion oligonucleotide;
b) partitioning the mixture obtained in step a) into several compartments so that a fraction of the compartments does not contain the target biomolecule;
c) converting the target biomolecule into a signal, said signal being preferably a DNA single strand;
d) amplifying the signal, and
e) detecting and/or measuring said amplified signal in each compartment.

The digital method according to the Invention allows to detect and/or quantify one or more biomolecules simultaneously, said biomolecules having the same or different structure and function.

In the context of the present invention, the term “biomolecule” relates to large macromolecules or biopolymers such as proteins, carbohydrates, lipids, and nucleic acids, as well as small molecules such as primary metabolites, secondary metabolites, and natural products. The biomolecules in the present invention are usually endogenous but may also be exogenous (for example, biopharmaceutical drugs).

According to one preferred embodiment of the present invention, the biomolecules are selected from the group comprising proteins, preferably enzymes or from nucleic acid.

The inventors surprisingly found that coupling enzymatic activities to a generic molecular circuit that performs thresholded exponential amplification of a DNA signal in digital bioassay allows to detect and/or quantify the enzymes with very high sensitivity compared to others digital enzyme test of the art (allowing the detection of few types of enzymes). The circuit includes a module, which connects the target activity to the generation of a short DNA trigger, and a DNA amplification system, which produces a detectable readout, particularly a fluorescent readout. Because it decouples the enzyme catalytic rate from the signal generation, this versatile framework allows for digital detection, particularly for droplet digital detection of a wide range of DNA processing enzymes using standard microcompartments, particularly microfluidic droplets with an inner volume in the picolitre range. The method of the invention thus allows that a weak catalytic activity associated with single enzymes is converted into detectable signals, particularly fluorescent signals strong enough to be easily detected inside microcompartments, particularly microdroplets.

In the context of the present invention the term “enzyme” designs proteins having catalytic function and which are able to catalyze the transformation of a molecular substrate. The groups of enzymes which may be detected and/or quantified by the digital method of the present invention are selected from DNA-related enzymes with a wide range of activities such as nucleases, DNA N-glycosylases, polymerases, ligases and kinases or non-DNA related enzymes. Particularly, the enzymes are selected from the group comprising nicking enzymes, restriction enzymes, endonuclease, DNA N-glycosylase, AP-endonuclease, exonuclease RNA/DNA polymerase, ligase, kinase and methylase. More particularly, the method of the invention may be implemented for identifying and/or quantifying nicking enzymes and restriction endonuclease. Specific examples of enzymes which may be detected and/or quantified by the method of the invention include but are not limited to Nt.BstNBI, RNAseH2. APE-endonuclease 1 (APE-1), uracile DNA glycosylase (UDG), alkyl adenine glycosylase (AAG), BsmAI restriction enzyme, poly(A) polymerase (PAP), T4 DNA ligase and T4 Polynucleotide Kinase (T4 PNK).

In the context of the present invention the term “module” relates to an oligonucleotide or a group of oligonucleotides performing a specific action, such a signal conversion, amplification, reporting, etc. In the case when nucleic acids are detected, the module corresponds to an oligonucleotide (a template). When the enzymes are detected the module corresponds to a group of oligonucleotides.

In another embodiment, the biomolecules targets of the present invention are nucleic acid such as DNA, cDNA, RNA, mRNA, microRNA. Even more preferably they are ribonucleic acids (RNA).

In the context of the present invention, the term “nucleic acids” relates to biopolymers or small biomolecules composed of nucleotides, which are monomers made of three components: a 5-carbon sugar, a phosphate group and a nitrogenous base. If the sugar is a compound ribose, the polymer is RNA (ribonucleic acid); if the sugar is derived from ribose as deoxyribose, the polymer is DNA (deoxyribonucleic acid).

As mentioned above, the digital method of the present invention may be used for detecting and/or quantifying DNA molecules or complementary DNA (cDNA) which is coding molecule.

Even more preferably the method of the present invention is used for detecting and/or quantifying ribonucleic acids (RNA) molecules selected from messenger RNA (mRNA), small interfering RNA (siRNA) and microRNA (miRNA).

According to the most preferred embodiment, the method of the present invention may be used to detect and/or quantify microRNA molecules.

In the context of the present invention, the term “microRNA” designs endogenous short non-coding RNA strands, with a length around 22 nucleotides involved in the post-transcriptional regulation of gene expression.

In order to implement the method of the present invention it is necessary in the first step (step a)) to mix the molecular program allowing to avoid background amplification with the biological sample containing the targeted biomolecule.

As used herein, the terms “target biomolecule” or “biomolecule target” relates to the biomolecules as defined above which is detected and/or quantifying by the method of the invention.

These target biomolecules are present in a sample. Said sample may be of any type. For example, said sample may be obtained from a tested subject, said subject being an animal, preferably a mammal and more preferably a human. Such sample may be also called a biological sample.

In the context of the present invention, the term “biological sample” relates to a solid or fluid biological material obtained from a life organism. Solid biological samples may be such as cells, part of tissue (biopsy), whole tissue or organ.

Preferably, the sample used in the method of the present invention is a fluid sample.

In the context of the present invention, the terms “sample of biological fluid” or “fluid sample” relate to any sample obtaining from bio-organic fluids produced by a life organism.

The biological fluids are selected from the group comprising extracellular fluids, intravascular fluids, interstitial fluids, lymphatic fluids and transcellular fluids.

Particularly, the sample of biological fluid is selected from the group comprising blood and blood components, urine, saliva, etc.

In more preferred embodiment the sample of biological fluid is a sample of blood or a sample of blood components. “Sample of blood” or “sample of blood components” means the total blood or one of its components selected especially from the red cells fraction, white cells fraction, platelets, plasma or serum.

In another embodiment of the present invention, the sample may be obtained from a non-living organism. For example, said sample may be obtained from air, water, soil, alimentary product etc. The type of the sample depends of the application of the digital method of the invention. According to the present invention, the said sample contains or is susceptible to contain biomolecules.

As indicated above, the sample containing targeted biomolecule is mixed with the molecular program in step a) of the digital method of the invention.

The molecular program used in the method of the invention is disclosed in details in the international application WO2017140815 which content is incorporate herewith by reference.

In the context of the present invention, the term “molecular program” relates to designing of biomolecular circuits to perform information-processing tasks in vitro. The molecular program is based on the predictable Watson and Crick base-pairing which confers to DNA a unique programmability enabling the rational design of molecular circuits.

The molecular program used in the method of the present invention is based on those developed by some of the present inventors which consists in a versatile molecular programming language named PEN-DNA toolbox ((Polymerase Exonuclease Nickase-Dynamic Network Assembly), Montagne et al., 2011). The topology of the network is defined by a set of short oligonucleotides (templates). The network is interpreted by a mixture of enzymes (polymerase, exonuclease and nickase or restriction enzyme), which process the information fluxes by producing and degrading DNA strands, which in turn activate or inhibit other nodes of the network.

According to one embodiment of the digital method of the present invention, the enzyme in the mixture of step a) are selected from the group comprising polymerase, nicking enzyme, restriction enzymes and exonuclease. The polymerase, the nicking enzyme and the restriction enzyme can drive the isothermal amplification and the exonuclease can avoid saturation of the system.

Particularly, polymerase used in the digital method of the present invention is selected from the group comprising Bst 2.0 DNA polymerase, Bst large fragment DNA polymerase, Klenow fragment (3′->5′exo-) (also called Klenow DNA polymerase (Klenow(exo-) or Klenow polymerase)), Phi29 DNA polymerase, Vent(exo-) DNA polymerase, more particularly, the polymerase is Vent(exo-) DNA polymerase (purchased from New England Biolabs (NEB)). According to one embodiment of the method of the present invention, more than one polymerase can be used simultaneously. According to a preferred embodiment polymerases Vent(exo-) DNA polymerase and Klenow fragment (3′->5′exo-) are used together. The concomitant use of Klenow polymerase with Vent(exo-) DNA polymerase allows to increase the speed of the amplification reaction, particularly when the detected biomolecule is RNA. The increase of the speed is essentially due to Klenow polymerase but the use of Vent(exo-) DNA polymerase remains necessary to avoid that unspecific amplification products appear.

In order to obtain an increased speed of the amplification reaction, the concentration of Klenow polymerase is comprised between 1-50 u/mL, particularly between 8 and 25 u/mL and even more particularly, this concentration is limited at 16 u/mL.

The nicking enzyme is selected from the group comprising Nb.BbvCI, Nb.BstI, Nb.BssSI, Nb.BsrDI particularly, the nicking enzyme is Nb.BsmI and/or Nt.BstNBI (purchased from New England Biolabs (NEB)). More than one nickase can be used simultaneously.

According to one embodiment of the method of the invention, the nicking enzymes may be replaced by restriction enzymes. Contrary to the nicking enzymes which cut the one strand, the restriction enzymes cut the double strand. Thus, when using restriction enzymes instead to nicking enzymes, it is necessary to protect the templates used in the method of the invention. This protection may be obtained for example by chemical modification of the templates. Such modifications comprise backbone modification such as phosphorothioate linkage.

The exonuclease used in the digital method of the invention is selected for example from RecJ_{f}, Exonuclease I, Exonuclease VII, particularly, the exonuclease is ttRecJ exonuclease obtained following the protocol described by Yamagata (Yamagata et al., 2001). More than one exonuclease can be used simultaneously.

The buffer used for the mixture of enzymes and oligonucleotide is adapted to the selected oligonucleotides templates. The skilled artisan would be able to adapt conventional buffers to particular molecular design. The experimental part of the present application also gives examples of such buffers. For example in preferred embodiment, the reaction buffer comprises 20 mM Tris HCl pH 7.9, 10 mM (NH₄)₂SO₄, 40 mM KCL, 10 mM MgSO₄, 50 μM each dNTP, 0.1% (w/v) Synperonic F 104, 2 μM netropsin, 200 μg/mL BSA.

According to one embodiment, particularly when enzymes are detected and/or quantified, the mixture of step a) comprises the use of at least one module.

Each oligonucleotide or module of the mixture of step a) of the digital method of the present invention has specific function finally allowing the conversion of the target biomolecule and the amplification of the obtained signal without inducing at the same time a background amplification, i.e. a signal amplification reaction happening in absence of the targeted biomolecule.

This mixture thus comprises a first oligonucleotide or a first module which is an amplification oligonucleotide, a second oligonucleotide or a second module which is a leak absorption oligonucleotide and a third oligonucleotide or a third module which is a target-specific conversion oligonucleotide. Particularly, the mixture comprises a third module for detecting and/or quantifying enzymes.

In the context of the present invention, the term “amplification oligonucleotide” or “autocatalytic template” or “aT” designs an oligonucleotide which is able to exponentially amplifies the trigger sequence. The examples of oligonucleotides which may function as amplification oligonucleotides are given in table 1 below.

In the context of the present invention, the term “leak absorption oligonucleotide” or “pseudotemplate” or “pT” relates to the oligonucleotide which binds the amplified sequence more strongly than the autocatalytic template but only adds a few nucleotides at its 3′ end and thus deactivates it for further priming on the autocatalytic template (because its 3′ end is now mismatched on the autocatalytic template). The leak-absorption oligonucleotide allows to avoid nonspecific amplification, also called herein background amplification (i.e. the amplification occurring in absence of the amplified signal sequence). It drives the deactivation of the trigger synthesized from leaky reactions. The pseudotemplate needs to be protected against degradation, exactly like the autocatalytic template is. This is done using a few phosphorothioates modifications at its 5′ end (or other exonuclease-blocking possibilities such as biotin-streptavidin modifications, inverted or modified nucleotides). Examples of leak absorption oligonucleotide are given in table 1 below.

In the context of the present invention, the term “target specific conversion oligonucleotide” or “conversion template” or “cT” relates to an oligonucleotide which converts the target biomolecule to a universal trigger sequence (or also called herein “signal” or “signal sequence”). The conversion template may or may not be protected against degradation, like the autocatalytic template is. The term ““conversion module” relates to a group of conversion oligonucleotides as defined herein.

According to one embodiment of the digital method of the invention, the first oligonucleotide includes a partial repeat structure containing a nicking enzyme recognition site, and the second oligonucleotide is able to bind, extend, deactivate and slowly release the products of polymerization along the first oligonucleotide, thereby inducing a threshold.

According to another embodiment of the digital method of the invention, particularly when the target is DNA or RNA sequence, a 3′ side of the third oligonucleotide can bind to a target sequence and upon polymerization and nicking, the third oligonucleotide outputs a sequence able to activate the first oligonucleotide above the threshold adjusted by controlling concentration of the second oligonucleotide.

According to one embodiment of the digital method of the invention, a 3′ end of the first oligonucleotide has a reduced affinity for an amplified sequence.

In still another embodiment, a 3′ end of the second oligonucleotide is complementary to the sequence amplified by the first oligonucleotide, and a 5′ end of the second oligonucleotide serves as a template to add a deactivating tail to the amplified sequence.

In one embodiment for implementing the present digital method, the concentrations of the first and second oligonucleotides are selected so that a reaction of the first oligonucleotide is faster than the reaction of the second oligonucleotide at high concentration of the amplified sequence but the reaction on the second oligonucleotide is faster than the reaction of the first oligonucleotide at low concentration of the amplified sequence, thereby effectively eliminating amplification unless the stimulus threshold is crossed.

In another embodiment the third oligonucleotide (conversion template or module) may be designed in different manner in function of the nature of the detected and/or quantified biomolecule. Particularly, when enzymes are detected, the conversion template (module) may be designed at different manners depending on the nature of the enzyme to be detected. For example, RNASe H and AP-endonuclease (APE-1) may be detected by introducing in the stem structure of the sensing template a ribonucleotide and an abasic site (AP), respectively. Uracil DNA glycosylase (UDG) may be detected for example by substituting the AP-site by a deoxyribouridine moiety. The detection of restriction enzymes may be achieved by appending the recognition site to the 5′ part of the sensing template. The conversion template may be designed to serve as substrate of the targeted enzyme, and include a specific sequence or one or more chemical modifications such as phosphate modifications, modified nucleobase, sugar or backbone moieties or structural features such as wobbles, mismatches, blunt or dangling extremities, flap sequences.

In still another embodiment, more than one conversion oligonucleotide may be used, particularly for detecting and/or identifying enzyme. These templates may be designed as described above. The use of a module composed of two or more oligonucleotide is possible when for example, the target enzyme is specific for double-stranded DNA substrate, or when the target enzyme uses multiple nucleic acid strands as substrate. These enzymes include some DNA N-glycosylases (e.g. Alkyl adenine glycosylase) or DNA or RNA ligases or kinases, transposases, DNA or RNA nucleases). Additionally, it is possible to design the conversion module so that the target enzyme triggers a cascade of modification that eventually leads to the production of trigger, initiating of the amplification. As an example, the enzyme poly(A) polymerase (PAP) can be detected and quantified using two oligonucleotides, the first one serves as a substrate for the polyadenylation, the second as a template to produce trigger outputs using the newly-polymerized poly(A) tail as an input.

According to one embodiment of the digital method of the present invention fourth oligonucleotide, which is a reporting probe, is added.

In the context of the present invention, the term “reporting probe” or “reporting template” or “rT” relates to an oligonucleotide which translates the presence of the trigger sequence into a detectable signal. Such detectable signal refers for example to particles aggregation, media jellification, an electrochemical signal or a chemiluminescent signal, preferably a fluorescent signal. Thus, the reporting probe is preferably a fluorescent probe.

In one embodiment, the reporting probe detects the signal strand amplified by the amplification oligonucleotide.

In yet another embodiment, the reporting probe is a self-complementary DNA strand modified at both extremities by a fluorophore and/or a quencher. As used herein, the term “self-complementary” means that two different parts of the same molecules can hybridize to each other due to base complementary (A-T and G-C). In the case of the invention, the two extremities of the single strand probe (a few nucleotides on the 3′ and 5′ part) can hybridize to each other and induce the quenching of the fluorophore by the quencher. The amplified sequence can bind to a portion of the reporting template, leading to the opening of stem-loop structure and thus the enhancement of the fluorescence signal.

The reporting probe may also comprise a loop which includes a nicking recognition site.

Compared to PCR probes (used for detecting) used in the prior art which must be designed (sequence) and optimized (length) for each target, the reporting probes used in the method of the invention are modular, meaning that they can be used for any target through signal conversion by the proper bistable module (autocatalytic template+pseudotemplate).

Examples of different oligonucleotides (templates) used in the step a) of the digital method of the invention are given in table 1 below and may be also given in the Example section of the application.

TABLE 1
Examples of oligonucleotides (templates).

| SEQ ID | Name of the | | |
|---|---|---|---|
| NO: | sequence | Nucleotides Sequence | Function |
| SEQ ID | CBe12PS3 | C*G*A*TCCTGAATG- CGATCCTGAATG-p | autocatalytic |
| NO: 1 | | | template |
| SEQ ID | CBe12-1P53 | C*G*A*TCCTGAATG- CGATCCTGAAT-p | autocatalytic |
| NO: 2 | | | template |
| SEQ ID | CBe12-2P53 | C*G*A*TCCTGAATG- CGATCCTGAA-p | autocatalytic |
| NO: 3 | | | template |
| SEQ ID | ptBe12T5SP | T*T*T*T*T-CGATCCTGAATG-p | pseudotemplate |
| NO: 4 | | | |
| SEQ ID | CBa12-1PS4 | C*T*C*G*TCAGAATGCTCGTCAGAAT-p | autocatalytic |
| NO: 5 | | | template |
| SEQ ID | ptBa12A4SP | A*A*A*ACTCGTCAGAATG-p | pseudotemplate |
| NO: 6 | | | |
| SEQ ID | ptBa12T5SP | T*T*T*T*T-CTCGTCAGAATG-p | pseudotemplate |
| NO: 7 | | | |
| SEQ ID | ptBa12T4S3P | T*T*T*T-CTCGTCAGAATG-p | pseudotemplate |
| NO: 8 | | | |
| SEQ ID | ptBa12T3S3P | T*T*T*-CTCGTCAGAATG-p | pseudotemplate |
| NO: 9 | | | |
| SEQ ID | ptBa12T2S3P | T*T*C*TCGTCAGAATG-p | pseudotemplate |
| NO: 10 | | | |
| SEQ ID | ptBa12T1S3P | r-C*T*CGTCAGAATG-p | pseudotemplate |
| NO: 11 | | | |
| SEQ ID | CBa12-2PS4 | C*T*C*G*TCAGAATG-CTCGTCAGAA-p | autocatalytic |
| NO: 12 | | | template |
| SEQ ID | CBa12P54 | C*T*C*G*TCAGAATG-CTCGTCAGAATG-p | autocatalytic |
| NO: 13 | | | template |
| SEQ ID | CBa12-1PS4 | C*T*C*GTCAGAATG-CTCGTCAGAAT-p | autocatalytic |
| NO: 14 | | | template |
| SEQ ID | CBa12-3PS4 | C*T*C*G*TCAGAATG-CTCGTCAGA-p | autocatalytic |
| NO: 15 | | | template |
| SEQ ID | pTk12T5S4P | T*T*T*T*T-CAATGACUCCTG-p | pseudotemplate |
| NO: 16 | | | |
| SEQ ID | ApTk12A1SUP | A*C*A*ATGACUCCTG-A-p | pseudotemplate |
| NO: 17 | | | |
| SEQ ID | ApTk12A2SUP | A*A*C*AATGACUCCTG-A-p | pseudotemplate |
| NO: 18 | | | |
| SEQ ID | ApTk12A3PS | A*A*A*-C*AATGACUCCTG-A-p | pseudotemplate |
| NO: 19 | | | |
| SEQ ID | ApTk12A4SUP | A*A*A*ACAATGACUCCTG A-p | pseudotemplate |
| NO: 20 | | | |
| SEQ ID | ApTk12A5SUP | A*A*A*AA CAATGACUCCTG A-p | pseudotemplate |
| NO: 21 | | | |
| SEQ ID | ApTk12A6SUP | A*A*A*AAA CAATGACUCCTG A-p | pseudotemplate |
| NO: 22 | | | |
| SEQ ID | Ck12-2PS4bioteg | bioteg*C*A*A*TGA CUC CTG CAA TGA CTC | Autocatalytic |
| NO: 23 | | C p | template |
| SEQ ID | Cba12-2b1ot3 | C*T*C*G*TCAGAATG CTCGTCAGAA bioteg | autocatalytic |
| NO: 24 | | | template |
| SEQ ID | ptBa12A5SP | A*A*A*A*A CTC GTC AGA ATG p | pseudotemplate |
| NO: 25 | | | |
| SEQ ID | Cbe12-254P | C*G*A*T*CCTGAATGCGATCCTGAA p | autocatalytic |
| NO: 26 | | | template |
| SEQ ID | ApTBe12A3S3P | A*A*A*CGATCCTGAATGAp | pseudotemplate |
| NO: 27 | | | |
| SEQ ID | CBe12-2noPS3 | C*G*A*TCCTGAATGCGATCCTGAA | autocatalytic |
| NO: 28 | | | template |
| SEQ ID | Ck12- | Bioteg CAA TGA CUC CTG CAA TGA CTC C p | autocatalytic |
| NO: 29 | 2S4noUbioteg | | template |
| SEQ ID | ApTk12A5S3P | A*A*A*AACAATGACUCCTGA p | pseudotemplate |
| NO: 30 | | | |
| SEQ ID | CBe12-2AU LP | C*G*A*TCCTGAATGCGATCCTGA | autocatalytic |
| NO: 31 | | | template |
| SEQ ID | D21tof5TBe12S3P | C*G*A*TCCTGAAAGCGAAGTTTGACTC | conversion |
| NO: 32 | | ATCAACATCAGTCTGATAAGCTA p | template |
| SEQ ID | CBe12-3noPS3 | C*G*A*TCCTGAATGCGATCCTGA | autocatalytic |
| NO: 33 | | | template |
| SEQ ID | D21tofBe12S0P | CGATCCTGAATG TCA ACA TCA GTC TGA | conversion |
| NO: 34 | | TAA GCT A p | template |
| SEQ ID | CBe12-2SPCy355 | Cy3.5 | autocatalytic |
| NO: 35 | | *C*G*ATCCTGAATGCGATCCATCCTGAA p | template |
| SEQ ID | CBc125PBMN35 | BMN3*C*A*G*TCCAGAATGCAGTCCAGAA p | autocatalytic |
| NO: 36 | | | template |
| SEQ ID | pTBc12T5SP | T*T*T*T*TCAGTCCAGAATG p | pseudotemplate |
| NO: 37 | | | |
| SEQ ID | 92atoF5TBe12PS0 | CGA TCC TGA AAG CGA AG T TTG ACT CAA | conversion |
| NO: 38 | | GCA TTG CAA CCG ATC CCA ACC p | template |
| SEQ ID | Let7atof5TBa12S0 | CTC GTC AGA AAG CGA AGT TTG ACT CAA | conversion |
| NO: 39 | P | ACT ATA CAA CCT ACT ACC TCA p | template |
| SEQ ID | CBa12-2AULP | C*T*C*GTCAGAATG CTCGTCAGAA A | autocatalytic |
| NO: 40 | | GCGAAGC p | template |
| SEQ ID | ApTBa12A3S3P | A*A*A*CTCGTCAGAATGA | pseudotemplate |
| NO: 41 | | | |
| SEQ ID | RPBe-Cy5 | bioteg UT TG DDQII CAT TCA ATT TIC | reporting probe |
| NO: 42 | | GAT CCT GAA TG Cy5 | |
| SEQ ID | CBe12-154bioteg | bioteg*C*G*A*TCCTGAATGCGATCCTGAAT p | autocatalytic |
| NO: 43 | | | template |
| SEQ ID | Cba12Sbioteg | bioteg*C*T*C*GTCAGAATGCTCGTCAGAATG | autocatalytic |
| NO: 44 | | p | template |
| SEQ ID | RPBa-Hex | biotin TTTTG BMNQ530 AATTCTATTTT CTC | reporting probe |
| NO: 45 | | GTC AGA ATT Hex | |
| SEQ ID | Cba12-3noPS3 | C*T*C*GTCAGAATG CTCGTCAGA | autocatalytic |
| NO: 46 | | | template |
| SEQ ID | RPBe-Cy5(2) | Cy5 *T*T*CAGGTTTTCGATCCTGAA BHQ2 | reporting probe |
| NO: 47 | | | |
| On peutt | RPBe-Cy5(3) | Cy5 *A*T*TCAGAATGCGATCCTGAAT BHQ2 | reporting probe |
| SEQ ID | | | |
| NO: 48 | | | |
| SEQ ID | ptBa12A6biot | biotin*A*A*AAAACTCGTCAGAATG p | pseudo-template |
| NO: 49 | | | |
| SEQ ID | 92atof1 Be12-3+3 | ATGCGATCCTGACGTTTGACTCAA GCA TTG | conversion |
| NO: 50 | | CAA CCG ATC CCA ACC | template |
| SEQ ID | Let7atof1Ba12- | ATGCTCGTCAGA CGT TTG ACT CAA ACT | conversion |
| NO: 51 | 3+3 | ATA CAA CCT ACT ACC TCA | template |
| SEQ ID | CBc12-3noPS3 | C*A*G*TCCAGAATGCAGTCCAGA | autocatalytic |
| NO: 52 | | | template |
| SEQ ID | RPBc-FAM | FAM*T*T*CTGG TTTTCAGTCCAGAA BHQ1 | reporting probe |
| NO: 53 | | | |
| SEQ ID | Atoα | C*A*G*T*CCAGAATGCAGTCCAGAA p | autocatalytic |
| NO: 54 | | | template |
| SEQ ID | pTα | T*T*T*T*TCAGTCCAGAATG p | pseudo template |
| NO: 55 | | | |
| SEQ ID | rTα | Atto633 *A*T*TCTGAATGCAGTCCAGAAT | reporting template |
| NO: 56 | | BHQ2 | |
| SEQ ID | Let7atoα | TGCAGTCCAGAAGTTTGACTCAAACTATACAA | conversion |
| NO: 57 | | CCTACTACCTCA p | template |
| SEQ ID | Let7ctoα | TGCAGTCCAGAAGTTTGACTCAAACCATACAA | conversion |
| NO: 58 | | CCTACTACCTCA p | template |
| SEQ ID | mir39toα | TGCAGTCCAGAAGTTTGACTCACAAGCTGATT | conversion |
| NO: 59 | | TACACCC p | template |

In the table 1 above, biotin and bioteg refer to biotinylated synthons, respectively using aminoethoxy-ethoxyethanol linker and the longer triethylene glycol linker. “*” denotes a phosphorothioate backbone modification and “p” designates a 3′ phosphate modification. In SEQ ID Nos: 16 to 23 and 30, thymidine (T) is replaced by deoxyuridine (U) in order to avoid the nicking of the product of polymerization of the trigger on the template. Atto633, FAM, Cy5, Hex, Cy3.5, BMN3 are fluorophores and BHQ1, BHQ2, BMNQ530 are quenchers.

On the basis of the sequences listed above and the examples described herein, various combinations of templates may be used for implementing the digital method of the invention The person skilled in the art, starting from the present description and general knowledge concerning nucleotide design will be able to determine other templates and other combination in order to implement the method of the invention for any target of interest.

Particularly, the selection of the sequence of templates depends on experimental parameters like the working temperature, the speed and specificity of the used enzymes, particularly of nickases. Preferably, amplification templates that contain the Nb.BsmI nickase site and templates that work with Nt.BstNBI may be used.

According to one embodiment of the method of the invention, the templates, preferably four templates (autocatalytic template, conversion template, pseudotemplate and reporting template) are linked by the sequence of the “universal trigger”, which is the amplified sequence.

FIG. 3 shows one embodiment of the connectivity of the circuit in the molecular program preferably used in the digital method of the present invention. The universal signal amplification part generates a bistable node, i.e. an amplification system that admits two states: a nonproductive state in absence of trigger, and a productive state once the amplification is initiated with a concentration of triggers exceeding a given threshold. This bistable node is composed of two templates: an autocatalytic template (aT), composed of a dual-repeat sequence catalyzing the exponential replication of preferably 12-mer oligonucleotide; a pseudo-template (pT) that absorbs the leak products stemming from nonspecific reaction on the autocatalytic template and therefore avoid background amplification. A conversion template (cT) is connected upstream to the aT: upon binding of the target to the input part of the cT, the conversion template catalyzes the production of a multiplicity of output strands, which in turn trigger the autocatalytic reaction on the aT. Downstream of the aT, a reporting template (rT) captures the amplified signal strands to produce a fluorescence signal. The detailed reaction network according to preferred embodiment of the method of the invention is presented in FIG. 2.

According to another embodiment the readout of the amplified signal may be performed not by using reporting template but for example, by DNA double strand insert (Evagreen or SYBR green), by aggregating nanoparticles or by forming particular DNA structures, DNAzymes, etc.

The step b) of the digital method of the present invention is performed by partitioning the mixture obtained in step a) into several compartments so that a fraction of the compartments does not contain the target biomolecule.

The partitioning of a sample is the basis of digital quantification methods. Digital quantification relies on partitioning of the sample containing the amplification mixture so that the target biomolecules are randomly distributed, in a such a way that just a few target biomolecules, preferably between zero and ten, more preferably between zero and five, even more preferably between zero and two and still even more preferably one molecules are present in most of the compartments. The random partitioning process generates a distribution of number of target biomolecule per compartment which follows a Poisson law. The digital quantification method can be applied when at least some compartments do not contain any target biomolecule. This fraction of empty compartments should be higher than 1%, preferably higher than 10%. Thus, according to the present invention, the most of compartments contain at least one target biomolecule but not all of these compartments.

In other words, the partitioning of the mixture obtained in step a) into several compartments is performed so that the distribution of the target biomolecules gives a number of compartments having received at least one biomolecule that is less than 100% of the total number of compartments.

As used herein, the term “digital method” or “digital amplification method” design a detection method, wherein the tested sample which contains the target biomolecule is portioned in several microcompartments such as the targets are randomly isolated in the compartments following a Poisson distribution. The reaction is subsequently carried out in each individual compartment allowing for the direct counting of discrete events and for absolute target quantification.

The partitioning of the sample may be performed by several means such as microfabricated chambers (such as for instance SlipChip or QuantStudio 3D from Thermofisher Scientific) or microdroplets (such as for instance QX200 system from Biorad or Naica System from Stilla technologies).

According to a preferred embodiment of the digital method of the present invention, the tested sample is partitioned into millions of water-in-oil droplets made by combining two immiscible liquids: the sample aqueous phase and the continuous hydrophobic phase such as undecane-1-ol, silicon oil, mineral oil, more preferably perfluorinated oils because of their high immiscibility with water, biocompatibility, low viscosity, transparency and general compatibility with microfluidic devices.

The mean size of the droplets is very important, since it determines the feasibility of the assay, the time required to perform the assay (corresponding to the time necessary for a droplet containing a single target to amplify) and the dynamic range of the assay (which is the range of concentration comprised between the lower limit of sensitivity of the assay and the highest concentration for which a significant fraction of the droplets do not turn on). Indeed, in a digital format it is necessary to adapt the size of the droplets so that the concentration corresponding to a single copy of the target encapsulated in one droplet reaches a value above the detection limit in bulk. If the droplets are too big, the typical sensitivity of the assay would not permit the detection, simply because the target is too diluted and would not trigger a signal distinguishable from the background. The size of the droplets must also be small enough (and hence the concentration of the single molecule in the droplet large enough) to ensure that the amplification is triggered in a reasonable time (typically, a few hours) for target-positive droplets. Smaller droplets will make the assay faster. Finally, given an appropriate size of droplets for the two conditions above, the dynamic range of a digital assay is comprised between the lower bulk target concentration for which the fraction of droplets that turn on is significantly higher than in the negative control, and the concentration for which a significant fraction of droplets do not turn on). Smaller droplets shift the dynamic range toward higher concentrations, while larger droplets, if compatible with the assay, shift its dynamic range toward lower concentrations.

In addition, some technical constraints apply on the size of the compartments or droplets. Smaller monodisperse droplets can be more difficult to produce, but they are typically more stable during high temperature incubation or cycling.

Most of the PCR-based digital assays of the art resort to droplets of 20 to 100 micrometers in diameter (i.e. from 4-500 pL). This is because of the high sensitivity of the PCR amplification method, which makes this method compatible with very low initial target concentrations. Isothermal techniques are less sensitive and thus are better suited to smaller size of droplets. Depending on the target of interest, the assays described herein achieve a sensitivity without droplet partitioning that is generally better than 1 μM and can achieve 1 fM in some cases, such as for enzyme detection. It is therefore preferable to use droplets that are relatively small for nucleic acid detection, particularly for microRNA detection, and that can be larger for enzyme detection. It is still possible to change the size of the droplets in order to shift the dynamic range of the assay.

In one embodiment, the size of the droplets is adapted so that a single copy of the target encapsulated in the volume of one compartment reaches a value around the picomolar. This also ensures that the amplification is triggered in a reasonable time (typically, a few hours) for target-positive droplets. If the compartments are too big, the typical sensitivity of the assay would not permit the amplification, simply because the target is too diluted.

The downsizing of the reaction compartments (from microliter scale in tube to picoliter scale in droplets) add stochastic effects in the amplification reaction (stemming from templates and enzymes distribution in droplets, surface effect, interactions between biomolecules and surfactant agent, inhomogeneity in temperature). These phenomena imply the dispersion of the droplets' amplification start time (i.e. all droplets containing a single target will not amplify at the exact same time). This dispersion is less prevalent for PCR-based reaction because the thermocycling process synchronizes the duplication cycles in-between all compartments. As a consequence of higher ON-time dispersion for isothermal reactions, it is important that empty droplets stay OFF until all target-containing droplets have turned ON. Classical isothermal methods (EXPAR, RCA, HCR etc.), while exhibiting good sensitivities in bulk, cannot deal with this constrain. It is a necessary requirement for isothermal digital approaches to use an method that provides a sufficient time window for a target concentration, together with compartments of a precisely selected size, such that a single copy of the target biomolecule, present in one compartment of this size, reaches the mentioned target concentration.

According to one embodiment, the droplets used in the digital method of the invention have a size comprised between 0.001 pL and 100 pL, preferably between 0.1 pL and 10 pL and more preferably between 0.5 and 5 pL or 0.5 to 8 pL. Preferably, when the method of the present invention is used to determine and/or quantify enzymes, the size of the droplet is is comprised between 5 and 8 pL, preferably, the size of the droplets in this case is 7.2 pL. According to another embodiment, when the method of the invention is used for detecting and/or quantifying enzymes, the size of the droplets may be comprised between 0.5 and 100 pL, particularly between 5 and 50 pL and more particularly, between 6 and 10 pL.

As indicated above, the main advantage of the method of the present invention is that the target biomolecule is not directly detected but is converted to a signal sequence. This allows to avoid the sensitivity and specificity issues caused by the direct detection of the target biomolecule and also limits the risk of carry-over contamination.

Thus, in the method of the present invention, after partitioning the mixture in step b), the sequence of target biomolecule is converted into a signal in step c), preferably by the conversion template.

As used herein, the term “signal” or “signal sequence” relates to nucleic acid sequence, preferably a single strand DNA which is obtained by converting the sequence of the target biomolecule, preferably nucleic acid molecule, more preferably, a microRNA into a sequence which may be amplified.

The converted signal sequence is then amplified in step d) of the method of the invention.

According to one embodiment, the amplification of signal sequence in step d) is performed at a constant working temperature ranging from 35 to 60° C., more preferably from 37 to 55° C. and even more preferably from 45 to 50° C.

According to the present invention, the chemical reactions in steps c) and d) occur at the same time. Steps c) and d) may be also designed as an incubation step.

In order to detect and/or measure the amplified signal sequence, it is necessary to label said molecule. Preferably the labelling is performed by using fluorescent probes Labelled with organic dyes or inorganic dyes such as quantum dots, particles agglutination. The skilled artisan would be able to adapt the conventional labeling means to the digital method of the present invention.

According to preferred embodiment of the digital method of the invention, the used label is a fluorescent signal. During the incubation for amplifying the signal sequence in step d), the target biomolecule triggers the signal amplification in its enclosing droplet. Following incubation, the droplet thus exhibits a positive fluorescence signal.

Thus, according to one embodiment of the digital method of the present invention, the step e) of detecting and/or measuring said amplified signal comprises detecting and/or counting the compartments emitting high fluorescence.

In one embodiment, when the digital method of the invention is used for measuring the absolute concentration of the target biomolecule in the tested biological sample, the compartments that display high fluorescence, which are those having received the target biomolecule, and the non-fluorescent compartments are counted and their ratio is calculated.

Preferably, the imaging by fluorescence microscopy permits the counting of positive/negative droplets and therefore the calculation of the exact target biomolecule concentration in the initial sample.

According to another embodiment, another conventional method, for example flow cytometry may be used for counting of positive/negative droplets.

According to the preferred embodiment, the digital method of the present invention is performed as follow: the mixture containing the molecular program (buffer, the above-mentioned enzymes, polymerase, nicking enzyme and exonuclease and four oligonucleotides templates mentioned above) and the target biomolecule-containing sample is partitioned into picoliter-sized (between about 0.1 and 5 pL) water-in-oil droplets using standard microfluidic techniques. The targets are randomly distributed in the droplets, following the Poisson law. The emulsion is then incubated at the constant working temperature (ranging from 42 to 55° C.) in order to allow the conversion of target biomolecule into signal and to amplify said signal. The signal is amplified and the fluorescence is turned on in the droplets which received at least one target biomolecule during the partitioning step. At the same time the empty droplets remain at a low fluorescence level. The droplets are finally imaged using fluorescence microscopy (other readout such as fluorescence activated droplet sorting (FADS) or flow cytometry might be used). The readout thus relies on an end-point measurement, by contrast to the real-time monitoring necessary for test tubes experiments. The ratio of positive/negative droplets (or also called “ON/OFF” ratio) allows the calculation of the exact target concentration in the initial sample without reference to a calibration standard.

The biomolecules mentioned above may be present in all type of sample. For example, they may be present in a sample obtained from non-living organism (for example soil sample, water sample, air sample, food sample etc) or in samples obtained from living organism, for example, cells, body fluids or tissues.

According to one embodiment of the method of the present invention, the target biomolecules detected and/or measured by the digital method of the invention are used as biomarkers.

In the context of the present invention, the term “biomarker” relates to a naturally occurring molecule, preferably a biomolecule, gene, or characteristic by which a particular pathological or physiological process, disease, etc. can be identified.

These biomarkers may be use for detecting diseases in living organisms such as plants, animals, preferably a mammal and more preferably a human.

Moreover, these biomarkers may be used for detecting one or several anomalies and food and agri-food industry or in the environment.

The biomarkers may be present for example in all bodily fluids and thus accessible via minimally invasive liquid biopsies (serum, plasma, urine, tears, saliva, sweat, etc).

Preferably, they are used as biomarkers for detecting diseases selected from the group comprising cancer, neuronal diseases, cardiovascular diseases, inflammatory diseases, autoimmune diseases, diseases due to viral or bacterial infection skin diseases, skeletal muscle diseases, dental diseases and prenatal diseases.

In the context of the present invention, the term “detection” is used for defining in general manner the detection of target biomolecule in a sample as defined above.

As used herein, the term “detection” also relates to the diagnosis or the prognosis of one or several of the above cited diseases or of their symptoms. The detection also includes the prediction of one or several of said diseases or of the prediction of the risk for a subject to develop one or several of these diseases.

Moreover, the term “detection” further relates to the agro-diagnosis, i.e. to the diagnosis of phytopathologies, particularly of phytopathologies having biotic or abiotic origin as defined in the present invention.

In the context of the present invention, the term “cancer” refers to a malignant neoplasm characterized by deregulated or uncontrolled cell growth. In particular, a “cancer cell” refers to a cell with deregulated or uncontrolled cell growth.

The term “cancer” includes primary malignant tumours (e. g., those whose cells have not migrated to sites in the subject's body other than the site of the original tumor) and secondary malignant tumors (e. g., those arising from metastasis, the migration of tumour cells to secondary sites that are different from the site of the original tumour). Such cancer may notably be selected from the group of solid cancers and/or from the group of hematopoietic cancers.

In one embodiment of the invention, the cancer is selected from osteolysis, bone sarcomas (osteosarcoma, Ewing's sarcoma, Giant cell tumours of bone), bone metastases, glioblastoma and brain cancers, lung cancer, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myleogeneous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukaemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer and Wilms' tumor.

In the context of the invention, the term “neuronal diseases” refers to the diseases of the central and peripheral nervous system comprising the brain, spinal cord, cranial nerves, peripheral nerves, nerve roots, autonomic nervous system, neuromuscular junction, and muscles. The neuronal diseases are selected from neurodevelopmental, neurodegenerative or psychiatric diseases. They include epilepsy, Alzheimer disease and other dementias, cerebrovascular diseases including stroke, migraine and other headache disorders, multiple sclerosis, Parkinson's disease, neuroinfections, brain tumours, traumatic disorders of the nervous system due to head trauma ect.

As used herewith “cardiovascular diseases” relate to heart or vessel failure including coronary heart disease: disease of the blood vessels supplying the heart muscle; cerebrovascular disease: disease of the blood vessels supplying the brain; peripheral arterial disease: disease of blood vessels supplying the arms and legs; rheumatic heart disease: damage to the heart muscle and heart valves from rheumatic fever, caused by streptococcal bacteria; congenital heart disease: malformations of heart structure existing at birth and deep vein thrombosis and pulmonary embolism: blood clots in the leg veins, which can dislodge and move to the heart and lungs.

As used herein, “inflammatory disease” preferably refers to acute pancreatitis; ALS; Alzheimer's disease; cachexia/anorexia; asthma; atherosclerosis; chronic fatigue syndrome, fever; diabetes (e.g., insulin diabetes); glomerulonephritis; graft versus host rejection; hemorrhagic shock; hyperalgesia, inflammatory bowel diseases; inflammatory conditions of a joint, including osteoarthritis, psoriatic arthritis and rheumatoid arthritis; ischemic injury, including cerebral ischemia (e.g., brain injury as a result of trauma, epilepsy, hemorrhage or stroke, each of which may lead to neurodegeneration); lung diseases (e.g., ARDS); multiple myeloma; multiple sclerosis; myelogenous (e.g., AML and CML) and other leukemias; myopathies (e.g., muscle protein metabolism, esp. in sepsis); osteoporosis; Parkinson's disease; pain; pre-term labor; psoriasis; reperfusion injury; septic shock; side effects from radiation therapy, temporal mandibular joint disease, tumor metastasis; or an inflammatory condition resulting from strain, sprain, cartilage damage, trauma, orthopedic surgery, infection or other disease processes.

In the context of the present invention “autoimmune diseases” are defined as conditions wherein the body of a subject produces antibodies directed against his own tissues and cells. Examples of autoimmune disease are: type I diabetes, Graves' disease, inflammatory bowel disease, multiple sclerosis, psoriasis, rheumatoid arthritis, systemic lupus erythematosus etc.

The diseases du to viral or bacterial infection are caused by pathogenic virus or bacterial strains. Example of such diseases are AIDS, ascariasis; athletes' foot; bacillary dysentery, chickenpox; cholera, common cold, dengue, diarrhea, diphtheria, filariasis, gonorrhea, herpes, hook worm disease, influenza flu, leprosy, measles, mumps, oriental sore, pinworm disease, plague, pneumonia, poliomyelitis, rabies, ringworm, septic sore throat, sleeping sickness, smallpox, syphilis, tetanus, typhoid, vaginitis, viral encephalitis, whooping cough, etc.

The “skin diseases” are conditions affecting the skin as for example acne, alopecia areata, basal cell carcinoma, bowen's disease, congenital erythropoietic porphyria, contact dermatitis, darier's disease disseminated superficial actinic porokeratosis, dystrophic epidermolysis bullosa, eczema (atopic eczema), extra-mammary paget's disease, epidermolysis bullosa simplex, erythropoietic protoporphyria, fungal infections of nails, hailey-hailey disease, herpes simplex, hidradenitis suppurativa, hirsutism, hyperhidrosis, ichthyosis, impetigo, keloids, keratosis pilaris, lichen planus, lichen sclerosis, melanoma, melasma, mucous membrane pemphigoid, pemphigoid, pemphigus vulgaris, pityriasis lichenoides, pityriasis rubra pilaris, plantar warts (verrucas), polymorphic light eruption, psoriasis pyoderma gangrenosum, rosacea, scabies, shingles, squamous cell carcinoma, sweet's syndrome, urticaria and angioedema, vitiligo

As used herein, the “skeletal muscular diseases” relates to diseases of bones, muscles (myopathy) and skeletal-muscular junction. For example, such diseases are selected from back pain, bursitis, fibromyalgia, fibrous dysplasia, Injuries to the growth plate, heritable disorders of connective tissue, Marfan syndrome, osteogenesis imperfecta, osteonecrosis, osteoporosis, Paget's disease of bones, scoliosis, spinal stenosis, tendinitis etc.

As used herein the “dental diseases” relate to dental and mouth issues. Example of such diseases are dental cavities, periodontal (gum) disease, oral cancer, oral infectious diseases, trauma from injuries, and hereditary lesions, etc.

As used herein the “prenatal diseases” relate to diseases that could impact pregnancy or fetal development such as AIDS, amniotic fluid, bleeding during pregnancy, cervix disorders, pregnancy diabetes, disseminated intravascular coagulation (DIC), ectopic pregnancy, erythroblastosis fetalis, fetal development issues, high blood pressure in pregnancy, HELLP syndrome, hydatidiform mole, hyperemesis gravidarum, intrauterine growth restriction, large for gestational age (LGA), miscarriage, placenta abruptio, placenta previa, placental insufficiency, polyhydramnios, prenatal testing, pregnancy loss, preterm labor and birth, rubella, small for gestational age (SGA), systemic lupus erythematosus, toxoplasmosis, twin-to-twin transfusion syndrome, twins, triplets, multiple births, vaginal bleeding during pregnancy etc.

The digital method of the present invention may be thus used in the in vitro diagnosis methods for diagnosing diseases selected from the group comprising cancer, neuronal diseases, cardiovascular diseases, inflammatory diseases, autoimmune diseases, diseases due to viral or bacterial infection skin diseases, skeletal muscle diseases, dental diseases and prenatal diseases.

In the second aspect, the present invention thus relates to an in vitro method for diagnosis of a disease selected from the group comprising cancer, neuronal diseases, cardiovascular diseases, inflammatory diseases, autoimmune diseases, diseases due to viral or bacterial infection skin diseases, skeletal muscle diseases, dental diseases and prenatal diseases. comprising the use of the digital method according to the invention.

According to one embodiment, said diagnosis method comprise the steps of:
providing a sample obtained from a subject, and
detecting the presence or the absence of one or more of said diseases by the use of the digital method of the invention.

The specificity, the sensitivity, the simplicity and the rapidity of the digital method of the invention allow to use it in agro diagnosis methods, particularly for diagnosis of diseases caused by biotic stress such as infectious and parasitic diseases, or caused by abiotic stress such as nutritional deficiencies or unfavorable environments.

According to the third aspect, the present invention also relates to an in vitro method for agro diagnosis of a disease selected from the group comprising:
diseases caused by biotic stress, preferably by infectious and/or parasitic origin, or
diseases caused by abiotic stress, preferably caused by nutritional deficiencies and/or unfavorable environment,
   said method comprising the use of the digital method of the invention.

According to one embodiment, said agro diagnosis method comprise the steps of:
providing a sample obtained from any one of plant's parts, and
detecting the presence or the absence of one or more of said diseases by the use of the digital method of the invention.

In the context of the present invention, the term “agro diagnosis” relates to a diagnosis of phytopathologies, said diagnosis comprising carrying out an analysis from plant samples for an identification of fungi, viruses, bacteria, nematodes and any other life organism causing biotic stress and/or for identifying biomolecules which presence is due to an abiotic stress.

In the context of the present invention, the term “phytopathology” or “plant disease” relates to plant anomalies which are manifested by changes in plant morphology, physiology or behavior due to a biotic or to an abiotic stress.

As used herein, the term “biotic stress” relates to stress or also to diseases caused by living organisms. The biotic stress may be caused by any living organism but has preferably an infectious and/or parasitic origin and may be caused by organisms selected from fungi, viruses, bacteria and nematodes.

According to one embodiment, the agro diagnosis method of the invention may be used for diagnosis of diseases caused by fungi, said diseases being selected from anthracnose, black knot, blight, chestnut blight (such as late blight), canker, clubroot, damping-off, Dutch elm disease, ergot, Fusarium wilt, Panama disease, leaf blister, mildew (such as downy mildew and powdery mildew), oak wilt, rot (such as basal rot, gray mold rot and heart rot), rust (such as blister rust, cedar-apple rust and coffee rust), scab (such as apple scab), smut, bunt, corn smut, snow mold, sooty mold and Verticillium wilt.

According to another embodiment, the agro diagnosis method of the invention may be used for diagnosis of diseases caused by viruses, said diseases being selected from curly top, mosaic, psorosis and spotted wilt.

According to still another embodiment, the agro diagnosis method of the invention may be used for diagnosis of diseases caused by bacteria, said diseases being selected from aster yellows, bacterial wilt, blight (such as fire blight and rice bacterial blight), canker, crown gall, rot, basal rot and scab.

The agro diagnosis method of the invention may also be used for diagnosis of diseases caused by nematodes selected from root-knot nematodes (such as Meloidogyne spp.), cyst nematodes (such as Heterodera and Globodera spp.), root lesion nematodes (such as Pratylenchus spp.), the burrowing nematode (such as Radopholus similis), Ditylenchus dipsaci, the pine wilt nematode (such as Bursaphelenchus xylophilus); the reniform nematode (such as Rotylenchulus reniformis), Xiphinema index, Nacobbus aberrans and Aphelenchoides besseyi.

According to one embodiment, the agro diagnosis method of the present invention is used for diagnosing diseases caused by “abiotic stress” which term defines the diseases which are not caused by living organisms. These diseases are preferably caused by nutritional deficiencies and/or unfavorable environment. For example, the abiotic stress may be caused by inappropriate pH, water availability (drought stress), temperature (heat stress and cold stress), oxygen and/or gases availability, mineral deficiencies (salinity stress) and toxics compounds (for example pollutants).

The specificity, the sensitivity, the simplicity and the rapidity of the digital method of the invention allow to also use it in methods for detection of biomolecules in the field of food, agri-food industry and in the environment. Particularly, the digital method of the present invention is used for detecting food, agri-food and environmental anomaly. This detection is performed by using the digital method of the invention for detecting biomolecules which may be considered as biomarkers for said anomalies.

Such biomolecules may be for example a part of living organism or may be produced by the activity of a living organism or also may be artificial biomolecules. These biomolecules (or biomarkers) are selected from the group comprising biopolymers, particularly, DNA, RNA, proteins and enzymes.

Said biomolecules are present in agri-foods and in foods in an original and/or in a transformed product.

They also may be present in the environment, for example, in the air, in the water and/or in the soil.

Thus, according to one aspect, the present invention also relates to in vitro methods for detecting biomolecules (biomarkers) in agri-food, in food industry and/or in environment, said methods comprising the use of the digital method of the invention.

In the context of the present invention, the term “food” relates to all food, basic or transformed, produced without using industrial process or by using such process.

In the context of the present invention, the term “agri-food”, relates to the agri-food industry, i.e. to the commercial production of food by farming.

The terms “environment” or “environmental» relate to natural environment, i.e an ecological units that function as natural systems without massive civilized human intervention, including all vegetation, microorganisms, soil, rocks, atmosphere, and natural phenomena that occur within their boundaries and their nature. These terms also relate to no natural or artificial environment which may be created by the human. According to one aspect, the present invention also relates to an in vitro method for detecting anomalies in a food and in an agri-food industry and/or in the environmental using the digital method of the invention.

Preferably, said method comprises:
providing a sample obtained from a food, from a agri-food or from the environment,
detecting a tested biomolecule (biomarker) in said sample by the digital method of the invention.

The inventors also demonstrated that the method of the invention may be used for detecting and/or quantifying different target biomolecules from different samples simultaneously or parallelly. For that, in one embodiment, the samples can be barcoded with a fluorescent dye for example so that the barcode fluorescence intensities of each droplet permit the assignment to each of the initial sample. The barcoding protocols are disclosed for example from Brouzes et al. (2009) and Genot et al. (2016). Thanks to this barcoding procedure, multiple samples can be emulsified, collected, incubated and analyzed simultaneously allowing thus reducing the detection time and cost.

Thus, according to the fourth aspect, the digital method of the present invention may be also used for assaying in parallel more than one sample, or more than one target biomolecules in a sample.

According to a preferred embodiment, in order to detect and/or quantify directly more than one biomolecule in one or more than one different sample even if the provided signal is weak, the inventors surprisingly found that the use of fifth oligonucleotide which is a crosstalk inhibiting oligonucleotide (called herein “killer oligonucleotide” or “killer template”) allows to significantly improve the specificity of the method. This embodiment relies on target-specific DNA circuits (comprising aT, cT, pT and/or rT) interconnected with DNA-encoded inhibitors that repress nonspecific signal amplification dues to the fact that the molecular crosstalks generate un-wanted communication between multiplexed detection channels.

In the context of the present invention, the “killer oligonucleotide” or “killer template” or “kT” thus relates to an oligonucleotide which is able to produce pseudotemplate (pT) of the opposite switch, thereby acting as a cross inhibitor for the amplification and mitigating unspecific crosstalk. The example of sequences for the killer template are given in the Table 1A below:

| SEQ ID NO: | αkB | C*A*T*TCTGGACTGAAAA-CAATGACTCGATCCTGAA p | [symbol] |
|---|---|---|---|
| 91 | | | |
| SEQ ID NO: | Bkα | C*A*T*TCAGGATCGAAAA-CAATGACTCAGTCCAGAA p | [symbol] |
| 92 | | | |
| SEQ ID NO: | αkBA0 | C*A*T*TCTGGACTG-CAATGACTCGATCCTGAA p | [symbol] |
| 93 | | | |
| SEQ ID NO: | BkαT4 | C*A*T*TCAGGATCGAAAA-CAATGACTCAGTCCAGAA p | [symbol] |
| 94 | | | |
| SEQ ID NO: | αkBA1 | C*A*T*TCTGGACTGT-CAATGACTCGATCCTGAA p | [symbol] |
| 95 | | | |
| SEQ ID NO: | αkBA2 | C*A*T*TCTGGACTGTT-CAATGACTCGATCCTGAA p | [symbol] |
| 96 | | | |
| SEQ ID NO: | αkBA3 | C*A*T*TCTGGACTGTTT-CAATGACTCGATCCTGAA p | [symbol] |
| 97 | | | |
| SEQ ID NO: | αkBA4 | C*A*T*TCTGGACTGTTTT-CAATGACTCGATCCTGAA p | [symbol] |
| 98 | | | |
| SEQ ID NO: | αkBA5 | C*A*T*TCTGGACTGTTTTT-CAATGACTCGATCCTGAA p | [symbol] |
| 99 | | | |
| SEQ ID NO: | αkBT4 | C*A*T*TCTGGACTGAAAA-CAATGACTCGATCCTGAA p | [symbol] |
| 100 | | | |

[symbol]

indicates data missing or illegible when filed

1 pM and 100 nM, preferably, between 10 pM and 20 nM and even more preferably between 100 pM and 10 nM in order to control the strength of the inhibition reactions. Therefore, according to this embodiment, the digital method of detecting and/or identifying biomolecules of the present invention, further comprises adding fifth oligonucleotide which is a cross inhibitory oligonucleotide for detecting and/or quantifying two or more biomolecules

The present invention also relates to a kit which may be used for detecting and/or quantifying target biomolecules accordingly to the methods of the invention.

According to one aspect, the present invention relates to a kit for detecting and/or quantifying at least one target biomolecule comprising:
a) a mixture of enzymes, preferably selected from the group comprising polymerase, nicking enzyme and exonuclease;
b) a mixture of oligonucleotides comprising a first oligonucleotide which is an amplification oligonucleotide, a second oligonucleotide which is a leak absorption oligonucleotide and a third oligonucleotide which is a target-specific conversion oligonucleotide and optionally a fourth oligonucleotide which is a reporting probe and
c) partitioning agent, preferably water-in-oil emulsion.

The parts of the kit, i.e. the enzymes, the oligonucleotides and the partitioning agent are as those defined above.

The kit of the present invention may also comprise instructions for use.

The particular embodiments of the present invention will appear clearly from the examples and the figures below.

### FIGURES

FIG. 1: Microfluidic chip design (scale bar represents 100 μm).

FIG. 2: Droplets analysis. a) water-in-oil droplets were sandwiched between two hydrophobic glass slides and imaged with an epifluorescence microscope. b) Atto633 fluorescence channel. c) bright field channel, d) brightfield channel defocused by a 10 μm offset. With this setting, the droplet outlines are reinforced and facilitate the droplets segmentation. e) The brightfield image (d) is binarized and f) all droplets are segmented using the morphological component command. g) The droplets are filtered according to their size and their circularity. h) The fluorescence of each droplet is extracted from a disk of radius r (with 3<r<6 pixels) and center xy (xy corresponding to the centroid of the selected component). i) The positive droplets correspond to the ones with a fluorescence exceeding a set threshold (here, threshold=7). Knowing the droplet volume, the concentration is calculated back from the Poisson law.

FIG. 3: Molecular program dedicated to the detection of microRNA. a) a 4-template DNA circuit encodes the connectivity of the molecular program, whose reactions are catalyzed by a set of enzymes (polymerase, exonucleases, endonuclease): the conversion template (cT) converts the target microRNA to a universal trigger sequence; the autocatalytic template (aT) exponentially amplifies the trigger sequence; to avoid nonspecific amplification (in absence of the microRNA), the pseudotemplate (pT) drives the deactivation of the triggers synthesized from leaky reactions; the reporting template (rT) uses the trigger sequences to generate a fluorescence signal. b) Real-time monitoring of the amplification reaction in presence of an increasing concentration of Let-7a. c) Correlation between the amplification time (Cq) and the concentration of Let-7a. Errors bars were calculated from three independent duplicate experiments.

FIG. 4: Detailed chemical reaction network of the molecular program for the detection of microRNA shown on FIG. 3.

FIG. 5: Bulk detection of Let-7a with a) the full molecular program, b) the molecular program without the converter template or c) without the pseudotemplate (pT). The amplification reaction is monitored in real-time and the amplification time (Cq) are plotted as a function of the Let-7a concentration.

FIG. 6: Droplet digital detection of microRNA. a) The sample is mixed with the molecular program and partitioned into millions monodisperse droplets resulting in the random distribution of the microRNA targets throughout the compartments. After incubation, the droplets are imaged by fluorescence microscopy. The droplets having received at least one target exhibit a positive fluorescence signal (1), while the others remain negative (0). b) Fluorescence snapshot of emulsified sample spiked with an increasing concentration of Let-7a after amplification. c) Analysis of 30000 droplets. d) Plot of the linear relationship between the expected (conc. th.) and the experimentally measured (conc. meas.) target concentration. e) Assaying others microRNA by adapting the converter template. f) Invention method specificity evaluated from the cross reactivity of Let7a over Let7c (1 mismatch) and Let7b (2 mismatches).

FIG. 7: Effect of the pseudotemplate and Nb.BsmI concentrations on the detection of Let7a. Samples containing a defined concentration of pT (from 0 to 15 nM) and Nb.BsmI (from 0.1 to 0.4 u/μL) are spiked with 0 or 1 pM Let-7a and the amplification reaction was monitored in real-time. a) Cq are plotted as a function of the pT and Nb.BsmI concentrations. MDS is plotted as a function of b) the pseudotemplate concentration and c) the Nb.BsmI concentration.

FIG. 8: Optimization of the Nt.BstNBI concentration. Samples spiked with 0 or 1 pM of Let7a are incubated in presence of a varying concentration of Nt.BstNBI. The Cq are plotted as a function of the Nt.BstNBI concentration.

FIG. 9: Molecular program to reduce false-positive droplets rate. Target-free samples with a varying concentration of pseudotemplate (pT) are partitioned into droplets. The fluorescence of the emulsions is monitored in real-time.

FIG. 10: Elimination of nonspecific amplification reaction. The molecular program with 0 or 15 nM of pT is spiked with 0 or 1 pM of synthetic Let-7a before partitioning. The droplets are incubated at 50° C. and the bulk fluorescence (emulsion-averaged) continuously monitored (solid lines). The incubation is stopped at different time points and the droplets imaged by fluorescence microscopy to extract the percentage of positive compartments (diamonds).

FIG. 11: microRNA detection from biological samples. a) Let7a detection from H1975 cell line. b) Let7a and mir-39ce detection from human colon total RNA.

FIG. 12: Effect of Klenow(exo-) on the detection of Let7a microRNA. a) Real-time amplification reaction in presence of 0 or 10 pM of Let7a and a varying concentration of Klenow(exo-). b) Amplification time (Cq) as a function of the concentration of Klenow(exo-). c) Experimental conditions.

FIG. 13: Digital detection of Let7a using a mixture of Klenow(exo-) and Vent(exo-). a) Measured concentration as a function of spike-in-expected concentration. b) Experimental conditions. Mix A (containing the enzymes) and mix B (containing the templates) are mixed on chip using a 3-inlet flow focusing device. Droplets are incubated at 50° C. for 200 minutes.

FIG. 14: Detection of cel-miR39 in plasma sample. Human blood samples were collected from healthy donors and plasma was obtained by centrifugation. 0 or 1 pM of cel-miR39 is spiked in an amplification mixture supplemented with 5% plasma (v/v) and RNAse inhibitor. The measured concentrations reported on the plot indicate the full recovery of the exogenous microRNA in 5% plasma.

FIG. 15: Conversion module designs and bulk detection of enzymatic activities (standard deviation calculated for at least three independent data points). a) Nt.BstNBI, b) RNAseH2. c) APE-endonuclease 1 (APE-1). d) Uracil DNA glycosylase (UDG). e) Alkyl adenine glycosylase (AAG). f) BsmAI restriction enzyme. g) Poly(A) polymerase (PAP). h) T4 DNA ligase. i) T4 Polynucleotide Kinase (T4 PNK).

FIG. 16: Digital detection of enzymes. a) Microscopy snapshots of 2D droplet arrays for 6 different concentration of Nt.BstNBI enzyme. b) Measured concentration (fM) as a function of the spiked-in concentration (u/mL) for different enzymes. The linear relationship demonstrates the absolute quantitativity provided by the digital readout. Error bars correspond to the 95% confidence interval on the measurement.

FIG. 17: Comparison of the digital assay quantification of Nt.BstNBI for small (0.95 pL) and big (7.2 pL) droplets. The concentrations computed in both experiments were consistent. Error bars correspond to the 95% confidence interval on the measurement.

FIG. 18: Tetrastable system built from two cross-inhibitory bistable switches. (a) Schematic of the tetrastable DNA circuit. Two microRNA sensing circuits (cT, aT, pT and rT) are interconnected by killer template αkβ and βkα, which repress unwanted cross activation. (b) Detailed mechanism of the five kinds of template (pol.=Vent(exo-), nick1=Nt.BstNBI, nick2=Nb.BsmI, RE=BsmI, exo=ttRecJ). Conversion templates (cT) convert the complementary microRNA target to a signal strand (α or β). Autocataytic tem-plates (aT) exponentially amplify the signal strands. Pseudotemplates, by deactivating a fraction of signal strands, suppress background amplification stemming from biochemical noise. Reporting templates (rT) transduce the molecular signal (α or β) to a detectable fluorescence signal (green=Oregon green fluorophore, red=Atto633 fluorophore). From the α or β strands, killer templates (kT) produce pT of the opposite switch, mitigating unspecific crosstalk. All produced strands are continuously degraded by the exo-nuclease to maintain the system dynamic. Only one half of the tetrastable circuit is represented here, the second half being obtained by substituting a by B and conversely.

FIG. 19: Killer templates efficiency. (a) The α switch, triggered by 10 pM if mir39, is connected to the killer template αkβ producing pTβ of different lengths (with a deactivating tail ranging from 0 to 5 adenylate moieties). (b) Fluorescence of the β switch (t=1000 min) as a function of the concentration of kT.

FIG. 20: Extended data from FIG. 19 (a) Amplification curves for different concentrations of αkβ producing pTβ of different lengths. (b) Cq plotted as the function of the αkβ concentration

FIG. 21: Determination of the kT concentration to suppress cross activation between the α and β switches. (a) A and B circuits are triggered with 0 or 10 pM of mir92a and let7a respectively in presence of an increasing concentration of αkβ and βkα. (b) Amplification time (Cq) of the α and β switches. (c) Color-coded representation of the Cq as a function of the concentration of kT. The dashed blue frames represent the concentration of kT for which the system reaches tetra-stability. (d) Amplification curves of the mir39/mir7 duplex assay (0 or 10 pM each target). (e) Measured Cq for 7 different duplex assays. The left inset represents the average Cq and standard deviation for the 7 assays

FIG. 22: Principle of the digital duplex assay. (a) Droplets were generated out of 4 samples (0 pM mir39/0 pM let7a, 3 pM mir39/0 pM let7a, 0 pM mir39/3 pM let7a, 3 pM mir39/3 pM let7a) with a microfluidic chip, and the emulsions were analyzed by microscopy. (b) 2D histograms of the probes' fluorescence (α switch=green fluorescence—mir39, β switch=red fluorescence—let7a). Vertical and horizontal dashed lines indicate the positive threshold for the α and β switch respectively (c) Histograms of the measured versus expected target concentrations. (d) Digital duplex assays of various samples. of different compositions (microRNA target and concentrations).

FIG. 23: In-solution singleplex versus duplex assay. (a) Amplification curves of α (top) and β (bottom) bistable circuits incubated separately with the amplification mix and 0 or 3 pM of let7a and mir39 targets (singleplex assay). (b) Amplification curve of α and β switches embedded in the full tetrastable circuit (duplex assay). (c) Amplification time measured for a duplicate experiment. From these data, it was concluded that the killer templates have little effect on the amplification time in these conditions.

FIG. 24: Limit of the blank singleplex versus duplex assay. 4 samples are assembled as follow: sample A=a circuit only, 0 pM target, sample B=β circuit only, 0 pM target, sample C=α and β circuit, 0 pM target, sample D=α and β circuit, 1 pM target mir39 and let7e. (a) Amplification curves of the 4 samples in solution. (b) Composite image of a portion of the microfluidics chamber (brightfield, green and red fluorescence). (c) 2D histograms of the droplets fluorescence (α switch=green fluorescence, β switch=red fluorescence). (d) Percentage of positive droplets. The percentage of false positive droplets is qualitatively similar whether the assay is performed in singleplex or duplex (false positive α=1.1% singleplex and 1.1% in duplex, false positive α=0.25% singleplex and 0.28% in duplex).

### EXAMPLES

The below Examples aim to demonstrate that the present invention allow high sensitivity molecule detection by digitalizing an analog method of isothermal nucleic acid amplification.

### Example 1: Detection of microRNA by the Digital Method of the Invention

Methods and Materials

Chemicals: Oligonucleotides (templates and synthetic microRNA) were purchased from Biomers (Germany). The sequences were purified by HPLC and checked by matrix-assisted laser desorption/ionization mass spectrometry. Templates were designed according to the rules described by Montagne et al. (Montagne et al, 2011 and Montagne et al., 2016). The autocatalytic template (aT), pseudotemplate (pT) and reporting template (rT) are protected from the degradation by the exonuclease by 5′ phosphorothioate backbone modifications. A 3′ blocking moiety (phosphate group for aT, pT, cT and quencher for rT) is used to avoid nonspecific polymerization. Table 2 below recapitulates all the sequences used throughout the invention.

TABLE 2
Oligonucleotides sequences used throughout the invention. “*” denotes
phosphorothioate backbone modification. “p” denotes 3+-phosphate
modification. Upper and lower cases represent 2′-deoxyribonucleotide
and ribonucleotide, respectively. Trigger seq or trigger sequence
relates to the sequence amplified by the autocatalytic template.
The sequences SEQ ID NO: 61 to 66 are also cited as sequences 54 to
59 in table 1.

| SEQ ID No | ID | Sequence | Function |
|---|---|---|---|
| 60 | α | CATTCTGGACTG | Trigger seq |
| 61 | αtoα | C*A*G*T*CCAGAATGCAGTCCAGAA p | aT |
| 62 | pTα | T*T*T*T*TCAGTCCAGAATG p | pT |
| 63 | rTα | Atto633 *A*T*TCTGAATGCAGTCCAGAAT BHQ2 | rT |
| 64 | Let7atoα | TGCAGTCCAGAAGTTTGACTCAAACTATACAACCTACTACCTCA p | cT |
| 65 | Let7ctoα | TGCAGTCCAGAAGTTTGACTCAAACCATACAACCTACTACCTCA p | cT |
| 66 | mir39toα | TGCAGTCCAGAAGTTTGACTCACAAGCTGATTTACACCC p | cT |
| 67 | Let-7a | ugagguaguagguuguauaguu | microRNA |
| 68 | Let-7b | ugagguaguagguugugugguu | microRNA |
| 69 | Let-7c | ugagguaguagguuguaugguu | microRNA |
| 70 | mir39-ce | ucaccggguguaaaucagcuug | microRNA |
| 71 | Expar- | AACTATACAACCTACTACCTCAAACAGACTCAAACTATACAACCTA | DNA |
| | Let7a | CTACCTCAA | |
| 72 | mir92a | AGG UUG GGA UCG GUU GCA AUG CU | microRNA |

Nb.BsmI and Nt.BstNBI nicking enzymes, Vent(exo-) DNA polymerase and BSA were purchased from New England Biolabs (NEB). A 10-fold dilution of Nt.BstNBI was prepared by dissolving the stock enzyme in Diluent A (NEB) supplemented with 0.1% Triton X-100. The exonuclease ttRecJ was home-brewed expressed and purified by chromatography according to the protocol published by Yamagata (Yamagata et al., 2001). The enzyme is stored at 1.53 NM in Diluent A+0.1% Triton X-100. All the proteins were stored at −20° C.

Cell Culture. Human non-small cell lung cancer cell line, H1975, and colorectal cancer cell line, HCT116, cells were utilized for miRNA extraction. HCT116 cells were cultured in DMEM/F12 media supplemented with 10% FCS, 100 units/mL penicillin G, and 100 Ng/mL streptomycin. H1975 cells were cultured in a RPMI 1640 medium supplemented with 10% (v/v) FBS, 100 units/mL penicillin and 100 Ng/mL streptomycin. Cells were grown in a 5% CO₂ incubator at 37° C.

microRNA extraction: Human colon total RNA (Thermofisher Scientific) was aliquoted at 13 Ng/mL and stored at −20° C. before use. For cellular extraction, microRNAs were extracted from around 1×10⁶ cells using TaqMan® miRNA ABC Purification Kit (Applied Biosystems) following the kit instructions. Briefly, cells were resuspended in 50 μL of 1×PBS, and mixed with 150 μL Lysis Buffer. After the cell lysis step, 2 μL of 1 nM external control cel-miR-39-3p oligonucleotides (Biomers) is spiked into the prepared sample and vortexed, to evaluate the extraction efficiency. The target microRNA is captured using magnetic Human Panel beads and eluted in 100 μL of elution buffer.

Reaction mixture assembly: All reaction mixtures were assembled at 4° C. in 200 μL PCR tubes: the templates are mixed with the reaction buffer (20 mM Tris HCL pH 7.9, 10 mM (NH₄)₂SO₄, 40 mM KCL, 10 mM MgSO₄, 50 μM each dNTP, 0.1% (w/v) Synperonic F 104, 2 μM netropsin, all purchased from Sigma Aldrich) and the BSA (200 μg/mL). After homogenization, the enzymes are added (300 u/mL Nb.BsmI, 10 u/mL Nt.BstNBI, 80 u/mL Vent(exo-), 23 nM ttRecJ). Each sample was spiked with the microRNA target, serially diluted in 1× Tris-EDTA buffer (Sigma Aldrich) using low-binding DNA tips (Eppendorf). The samples (bulk or emulsion) were incubated at 50° C. in a qPCR thermocycler (CFX96 Touch, Biorad) and the fluorescence was recorded in real-time. For bulk experiments, the time traces were normalized and the Cq (amplification starting times) determined as 10% of the maximum fluorescence signal.

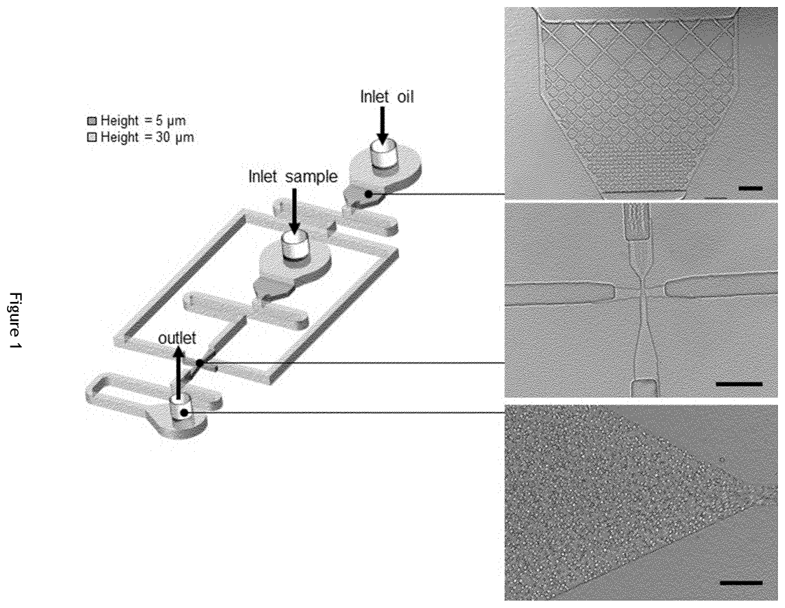

Droplets generation: a 2-inlet (one for the oil, one for the aqueous sample) flow focusing microfluidic mold was prepared with standard soft lithography techniques using SU-8 photoresist (MicroChem Corp., MA, USA) patterned on a 4-inch silicon wafer. A 10:1 mixture of Sylgard 184 PDMS resin (40 g)/crosslinker (4 g) (Dow Corning, MI, USA) is poured on the mold, degassed under vacuum and baked for 2 hours at 70° C. After curing, the PDMS was peeled off from the wafer and the inlets and outlet holes of 1.5 mm diameter were punched with a biopsy punch (Integra Miltex, PA, USA). The PDMS layer was bound onto a 1 mm thick glass slide (Paul Marienfeld GmbH & Co. K.G., Germany) immediately after oxygen plasma treatment. Finally, the chip underwent a second baking at 200° C. for 5 hours to make the channels hydrophobic (Kaneda et al., 2012). The microfluidic chip details are presented in FIG. 1. The aqueous sample phase and the continuous phase composed of fluorinated oil (Novec-7500, 3M) containing 1% (w/w) fluorosurfactant (RAN Biotechnologies, MA, USA) were mixed on chip using a pressure pump controller MFCS-EZ (Fluigent, France) and 200 μm diameter PTFE tubing (C.I.L., France) to generate 0.5 pL droplets by hydrodynamic flow focusing.

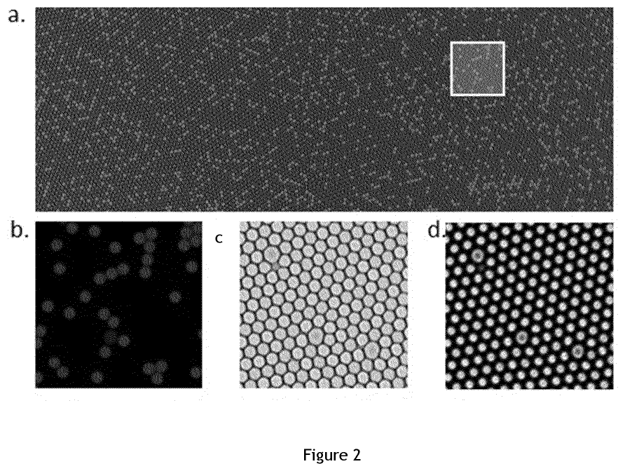

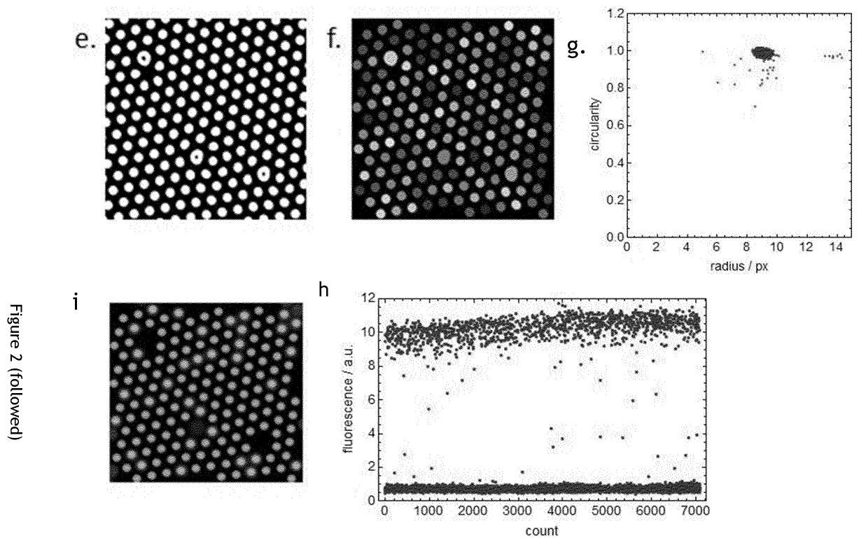

Droplets imaging: After incubation, the droplets were imaged by fluorescence microscopy. The bottom slide (76×52×1 mm glass slide) was spin-coated with 200 μL Cytop CTL-809M (Asahi Glass) and dried at 180° C. for 2 hours. The emulsion was sandwiched with a 0.17 mm thick coverslip treated with Aquapel. 10 μm polystyrene particles (Polysciences, Inc., PA, USA) were used as spacer to sustain the top slide and avoid the compression of the emulsion. The imaging chamber was finally sealed with an epoxy glue (Sader) and images were acquired using an epifluorescence microscope Nikon Eclipse Ti equipped with a motorized XY stage (Nikon), a camera Nikon DS-Qi2, an apochromatic 10× (N.A. 0.45) (Nikon) and a CoolLed pE-4000 illumination source. Composite images were generated with the open source ImageJ software. Quantitative data were extracted from the microscopy images using the Mathematica software (Wolfram), following the procedure detailed in FIG. 2.

Results

Analog Amplification Method According to the International Application WO2017140815

The inventors of the present invention previously developed a versatile molecular programming language named PEN-DNA toolbox (Polymerase Exonuclease Nickase-Dynamic Network Assembly) (Montagne et al., 2011 and Baccouche et l., 2014). The topology of the network is defined by a set of short oligonucleotides (templates). The network is interpreted by a mixture of enzymes (polymerase, exonuclease and nickase), which process the information fluxes by producing and degrading DNA strands, which in turn activate or inhibit other nodes of the network.

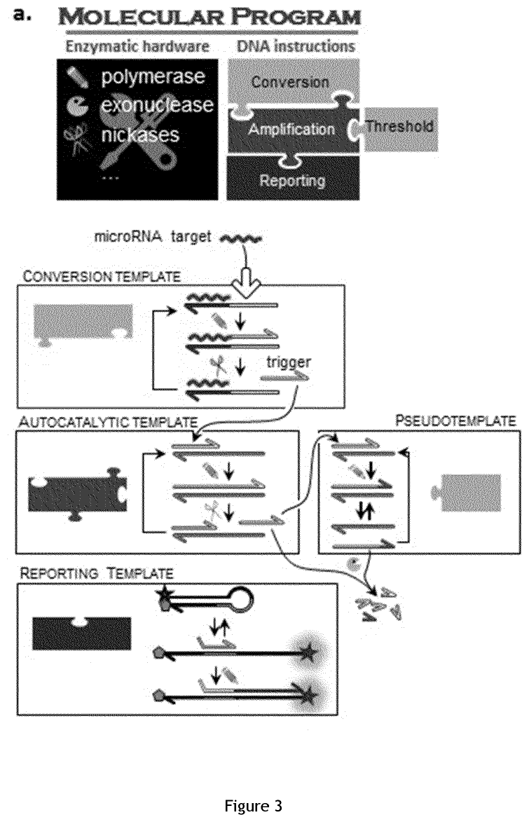

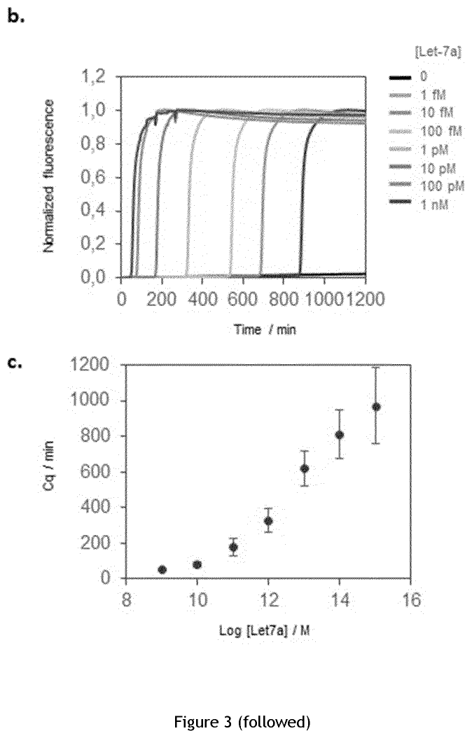

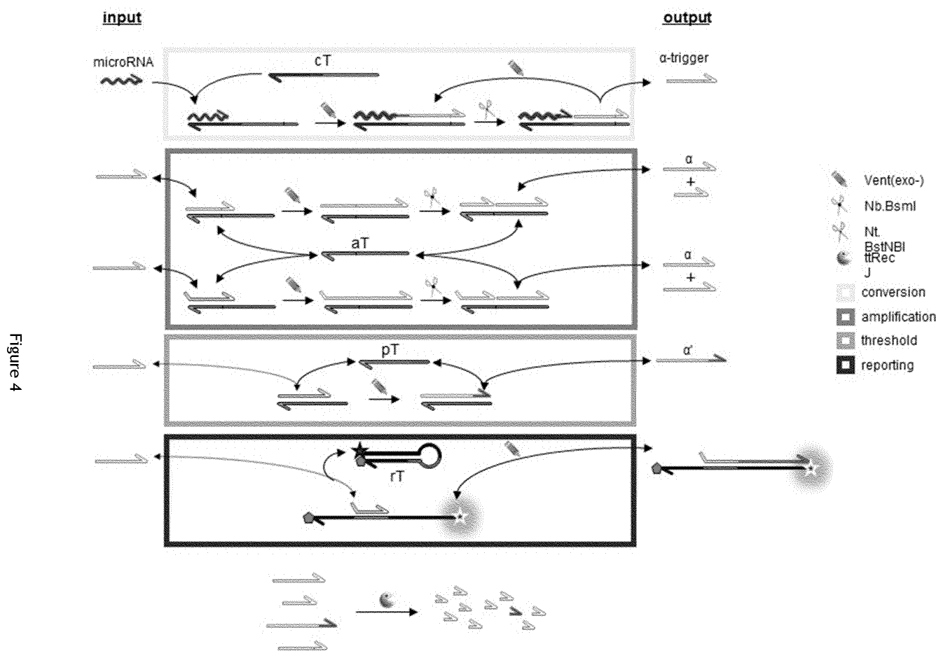

Using this set of reaction modules, the inventors previously designed a generic molecular program dedicated to the detection of microRNA. FIG. 3 presents the connectivity of the circuit: the universal signal amplification part corresponds to a bistable node composed of two templates: an autocatalytic template (aT), composed of a dual-repeat sequence catalyzing the exponential replication of a 12-mer oligonucleotide; a pseudo-template (pT) that absorbs the leak products stemming from nonspecific reaction on the autocatalytic template and therefore avoid background amplification. A converter template (cT) is connected upstream to the aT: upon binding of the target to the input part of the cT, the latter catalyzes the production of an output strand, which in turn triggers the autocatalytic reaction on the aT. Downstream to the aT, a reporting template (rT) captures the amplified signal strands to produce a fluorescence signal. The detailed reaction network is presented in FIG. 4.

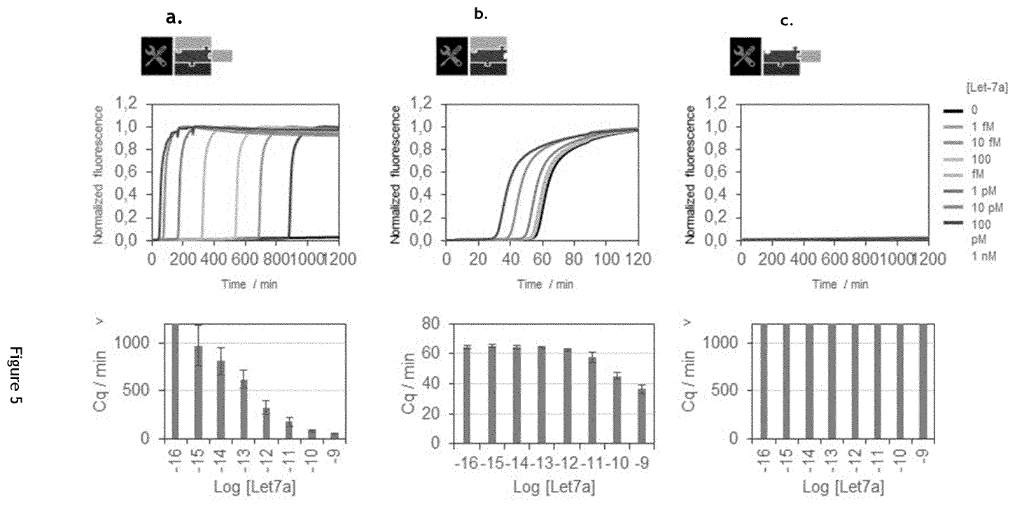

FIG. 5b-c show the evaluation of the sensitivity of this approach for the bulk detection of Let-7a. The 4 templates are mixed together with the enzymatic processor and spiked with a concentration of synthetic target Let-7a ranging from 0 to 1 nM. The fluorescence of the rT is monitored in real-time with a PCR thermocycler set at a constant temperature of 50° C. The negative control (no target) does not produce a positive signal for more than 20 hours. The sensitivity of the assay is around 1 fM and the dynamic range in bulk ranges from 1 fM to 100 pM, i.e. 6 orders of magnitude. In absence of the pT (FIG. 5b), the sensitivity is negatively affected with a limit of detection of 1 pM (estimated from 3 standard deviation from the mean amplification time of the negative control). These results demonstrate the importance of this active leak-absorption mechanism for controlling the amplification threshold and thus eliminating background amplification. In absence of the cT (FIG. 5c), the amplification reaction is not observed, demonstrating the specificity of the molecular program for the targeted microRNA.

Digitalization of the Analog Amplification Method

To convert the analog signal to a digital readout, the inventors move on to evaluate the possibility to detect single molecules compartmentalized in droplets. The molecular program spiked with a known concentration of Let-7a was partitioned in picoliter-sized water-in-oil droplets using a flow focusing microfluidic junction. The monodisperse emulsion was incubated at 50° C. and the reaction stopped after the target-containing droplets amplified. Finally, the droplets were imaged by fluorescence microscopy and the concentration recalculated from the Poisson law.

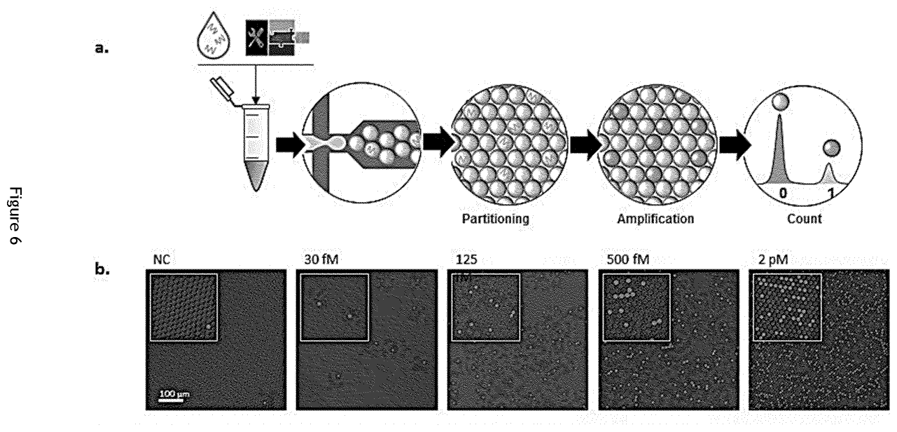

FIG. 6 shows the microscopy snapshots of the emulsions after incubation for 200 minutes. It is observed a linear correlation between the spiked miRNA concentration and the concentration measured according to the Poisson law. The calculated limit of detection is 2.1 fM.

The modular approach of the current system enables to repurpose the molecular program by redesigning only the converter template (FIG. 6d) to hybridize to any target of interest, without affecting the quantification, which depends on a universal signal amplification machinery.

A major concern related to the quantification of microRNA is the high sequence homology in between the microRNA targets. The inventors thus evaluated the specificity of the current detection method over the Let-7 family. FIG. 4e shows a very good discrimination between the Let-7a sequence and analogs containing a single (Let-7c) or dual (Let-7b) mismatched bases.

Effect of the Pseudotemplate (pT) and the Nuclease Concentration

The inventor also assayed the effect of the pseudotemplate (pT) and the nuclease concentration (Nb.BsmI).

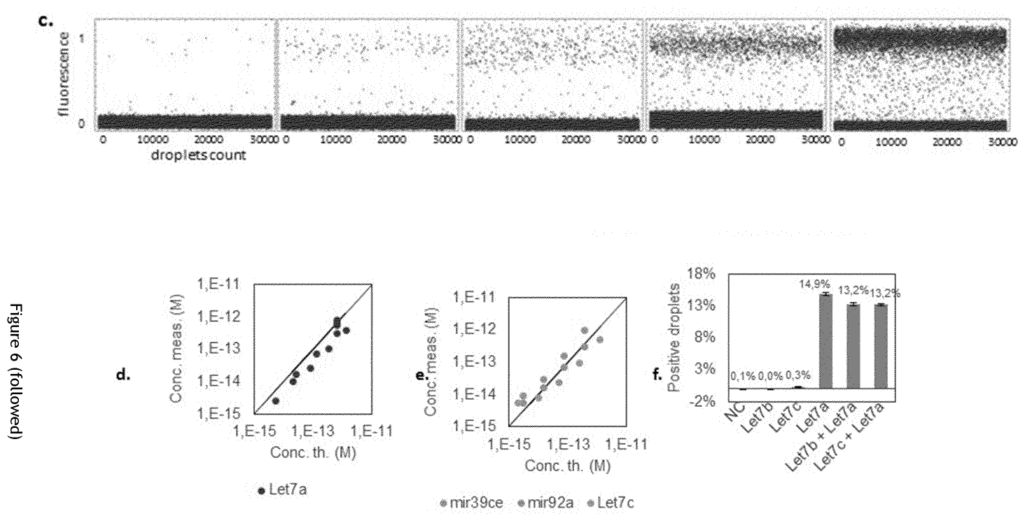

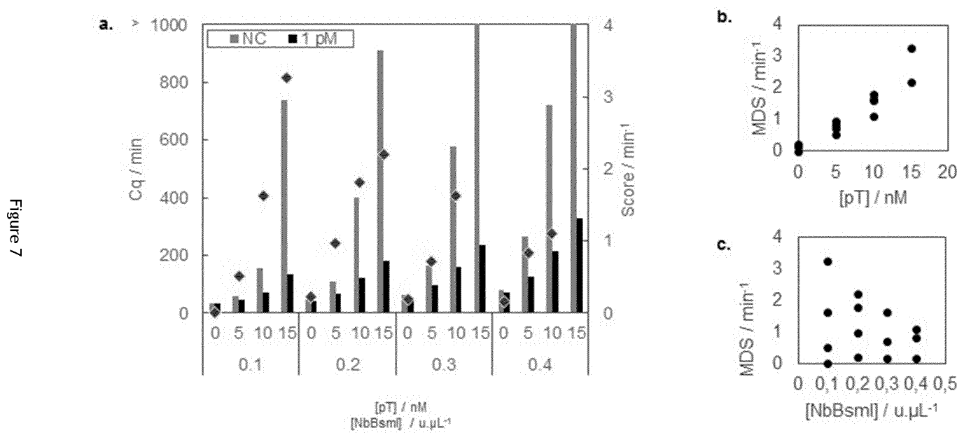

For that samples containing a defined concentration of pT (from 0 to 15 nM) and Nb.BsmI (from 0.1 to 0.4 u/μL) are spiked with 0 or 1 pM Let-7a and the amplification reaction was monitored in real-time. The Cq are plotted as a function of the pT and Nb.BsmI concentrations (FIG. 7a). A microRNA detection score (MDS) is calculated for each set of concentration according to the following equation. MDS=100(Cq_{NC}/Cq_{1 pM}−1)/Cq_{1 pM}. The MDS is meant to reflect both the time window between the amplification times of the NC and 1 pM and the speed of the target-triggered reaction. The MDS is plotted as a function of the pseudotemplate concentration (FIG. 7b) and the Nb.BsmI concentration (FIG. 7c).

FIG. 7 shows that both parameters delay the amplification reaction: the pseudotemplate plays the role of an active sink that degrades part of the produced triggers. Nb.BsmI is known to be inhibited by its own product (the nicked, un-melted duplex), which probably slows down the reaction by preventing the release of the trigger after cutting the duplex. It is to note that increasing the pseudotemplate concentration positively affects the detection score, by delaying preferentially the negative control (NC) amplification and affects to a lesser extent the target-triggered amplification. On the other hand, Nb.BsmI affects the amplification time, irrespective from the target concentration and thus negatively affect the detection score. This observation demonstrates the importance of the active degradation mechanism provided by the pseudotemplate to reduce/abolish background amplification even when the amplification method is digitalized.

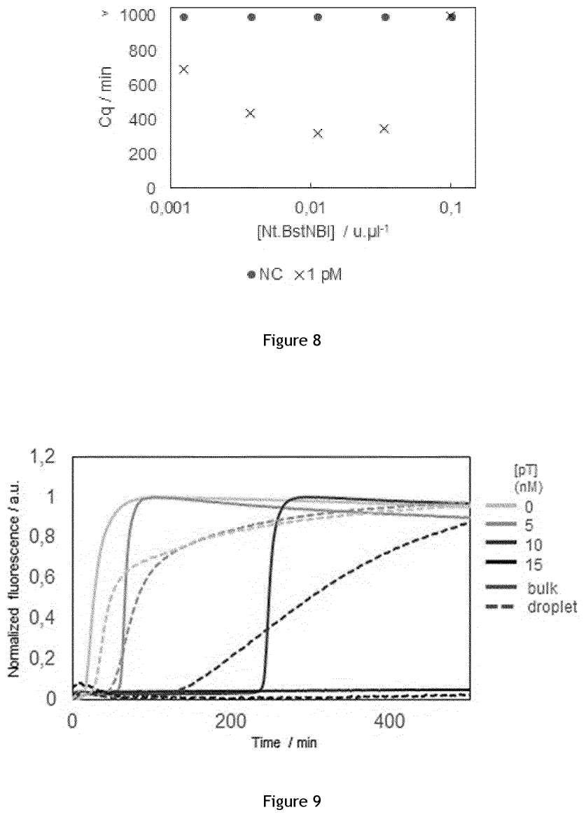

The inventors also investigated the optimal concentration of nuclease Nt.BstNBI. For that, the samples spiked with 0 or 1 pM of Let7a are incubated in presence of a varying concentration of Nt.BstNBI. The Cq are plotted as a function of the Nt.BstNBI concentration. FIG. 8 shows that it exists an optimal concentration of the endonuclease Nt.BstNBI around 0.01 u/μL.

### Comparative Example

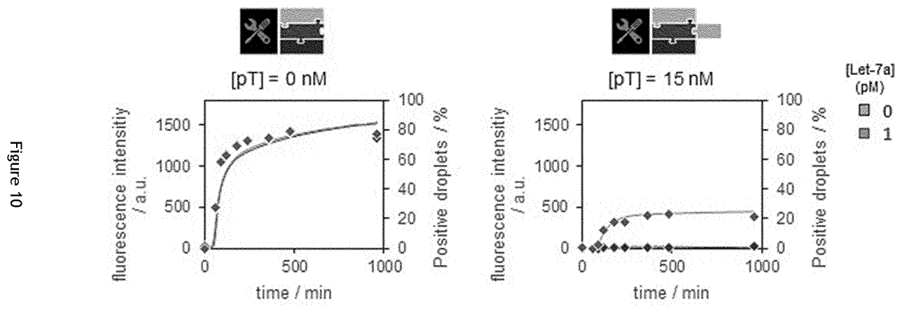

Most isothermal nucleic acid amplification techniques cannot be transposed to a digital format. This is generally attributed to nonspecific reactions, which eventually trigger the amplification in all compartments, irrespective of the presence of the target as described in Zhang et al. (Zang et al., 2015). Relying on an end-point analysis, it becomes crucial to have a time window large enough to discriminate the target-containing droplets (exhibiting a positive signal) from the target-free droplets. FIG. 9 shows that in absence of pT and target, all the droplets turn on in less than an hour. This result affects considerably the time-window required to separate the two populations and is consistent with a previously described EXPAR system described by Zhang et al. (Zhang et al., 2015).

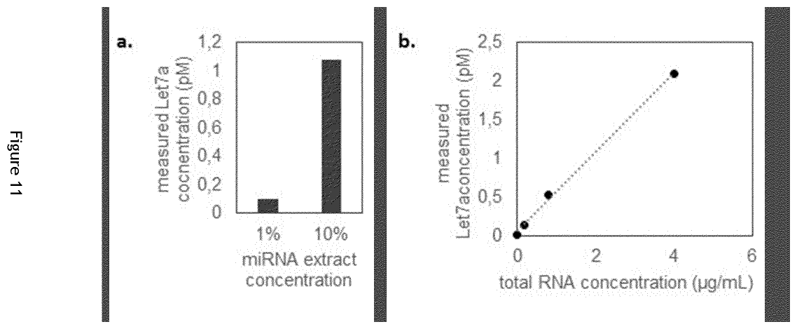

By increasing the pT concentration and thus raising the amplification threshold, the self-start is delayed until being completely cancelled at 15 nM of pT. FIG. 10 compares the time window for the detection of 1 pM of Let-7a with 0 or 15 nM of pT. In absence of the pT, it is nearly impossible to distinguished between the target-containing sample and the negative control. However, the absorption of the leak responsible for false positive droplets guarantees the stabilization of the off-state for more than 16 hours, without hampering the amplification of target-containing droplets. Thanks to the complete background elimination, the method of the present invention displays an unrivaled robustness with respect to incubation time and shows a theoretically infinite time window.

Moreover, compared to other digital amplification method previously implemented (Zhang et al., 2015, Cohen et al., 2016 and Tian et al., 2016), the selectivity of the method of the present invention is estimated to be superior to 97%.

Detection of Endogenous microRNA from Human Cells by the Method of the Invention

The success of microRNA-based diagnostic as a routine biomedical procedure depends on the robustness and reproducibility of the microRNA quantification. The inventors thus evaluated the possibility to detect endogenous microRNA from human cells.

microRNAs were extracted from the cell line H1975 (adenocarcinoma) and Let-7a was quantified by the method of the invention, with a varying concentration of the RNA extract. The measured Let-7a concentration from sample containing 1% and 10% of RNA extract are 90 fM and 1 pM respectively (FIG. 11a).

Additionally, the inventors quantified Let-7a from human colon total RNA: FIG. 11b shows the linear relationship between the total RNA concentration (ranging from 0 to 4 μg/mL) and the measured Let-7a concentration. As a negative control experiment, mir-39ce, absent from human genome, was not detected in these samples. Overall, this demonstrates the accuracy of the method of the invention and its robustness in high complex background samples.

### Example 2: Use of Klenow(3′→5′ Exo-) DNA Polymerase to Accelerate microRNA-Triggered Amplification

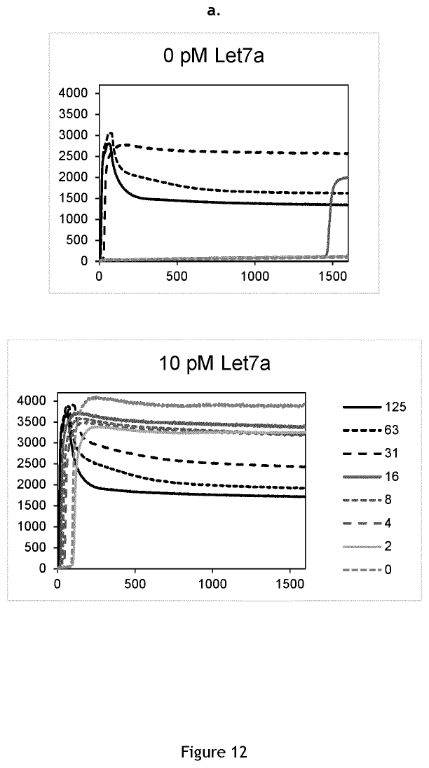

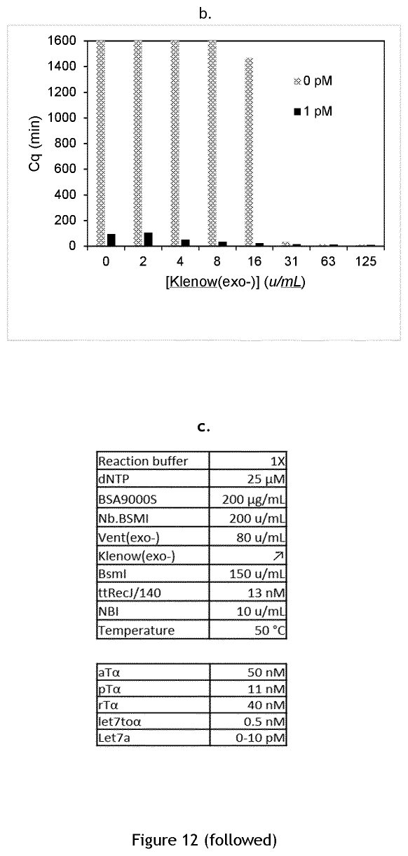

In order to accelerate microRNA targeted amplification, the inventors investigated the effect of adding another DNA polymerase which is Klenow(exo-). An amplification mixture (containing Vent(exo-) polymerase), spiked with 0 or 10 pM of Let7a microRNA target, is supplemented with a varying concentration of Klenow(exo-). FIG. 12 shows that in absence of Klenow(exo-) polymerase the specific amplification occurs in about 100 minutes, while the negative control does not amplify within 1000 minutes. Interestingly, the specific amplification is brought down to 20 minutes with 16 u/mL of Klenow(exo-), while the negative control is not affected. Above this concentration, the inventors observed undesirable self-amplification of the negative control samples in less than 40 minutes. Altogether, these results suggest that the addition of an optimal concentration of Klenow(exo-) in addition to Vent(exo-) is beneficial for accelerating the amplification reaction because Klenow(exo-) initiates efficiently the extension of the RNA primer.

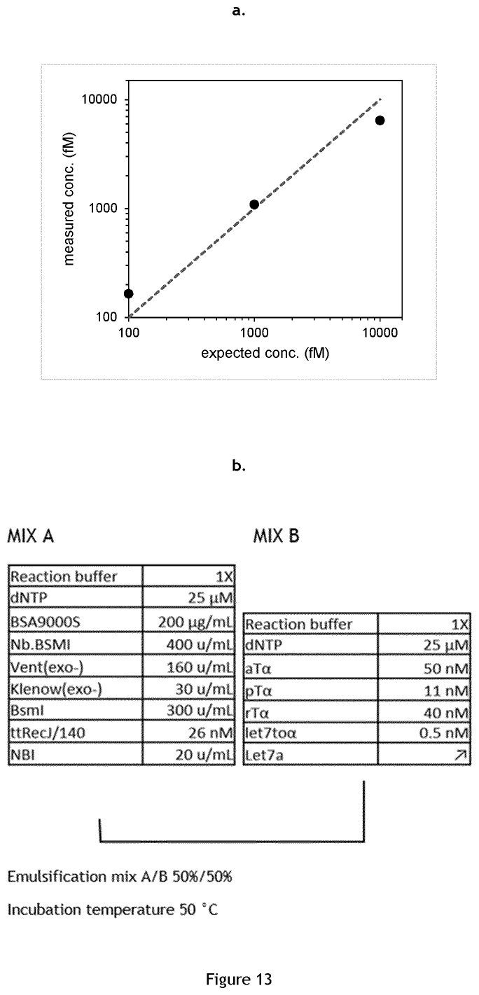

The inventors then investigated the use of a mixture of DNA polymerases for the droplet digital detection of microRNA. Since Klenow(exo-) possesses a non-negligible activity at room temperature, oligonucleotides (templates) and enzymes were assembled separately (mix A and mix B) and mixed on chip just before the droplet partitioning using a 3-inlet microfluidic device (one inlet for the continuous phase and 2 inlets for both parts of the amplification mixes A and B). This prevents the reaction from starting prior to target encapsulation. The measured concentrations are consistent with spike-in concentration of each sample, demonstrating the accurate digital quantification of the target microRNA with this polymerase mixture (FIG. 13).

### Example 3: Detection of microRNA Directly from Plasma Samples

The inventors also assessed the detection of microRNA in plasma samples using the digital detection method of the invention.

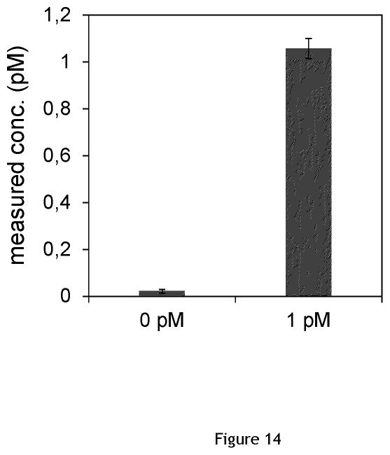

Human blood samples were collected from healthy donors (HIV, HBV, and HCV negative) into 10 mL blood collection tubes (Streck tubes supplied by Biopredic International). Plasma was obtained by centrifugation for 10 min at 2000×g at 4° C. followed by centrifugation for 15 min at 2000×g at 4° C. Plasma was aliquoted in clean polypropylene tubes using a Pasteur pipette, and stored at −80° C. until use. 0 or 1 pM of cel-miR39 (microRNA sequence from C. Elegans.) is spiked in an amplification mixture supplemented with 5% plasma (v/v) and RNAse inhibitor, murine at 1 u/μL. The measured concentrations reported on the plot indicate the full recovery of the exogenous microRNA in 5% plasma. The results show on FIG. 14 thus demonstrates the quantitative measurement of microRNA concentration in crude plasma samples.

### Example 4: Detection of Enzymes by Using the Digital Method of the Invention

Oligonucleotides were obtained from Biomers or Eurofins (Table 3). Nicking enzymes nt.BstNBI (R0607), Nb.BsmI (R0706), DNA polymerase Vent(exo-) (M0257), Klenow(exo-) polymerase, restriction enzyme BsmAI (R0529), AP-endonuclease APE-1 (M0282), Uracil DNA glycosylase UDG (M0280), Alkyl Adenine glycosylase hAAG (M0313), poly(A) polymerase (M0276), T4 DNA ligase (M0202), T4 polynucleotide kinase PNK (M0201) were purchased from New England Biolabs. RNAse H2 enzyme (11-03-02-02) was purchased from Integrated DNA technologies. The exonuclease ttRecJ was home purified according to previously reported procedure (8). All oligonucleotides and proteins were stored at −20° C.

TABLE 3
Sequences used in this study.“*” denotes phosphorothioate backbone
modification. “p” denotes phosphate modification. Upper and lower
cases represent 2′-deoxyribonucleotide and ribonucleotide,
respectively. “F” denotes an analog of abasic site, a tetrahydrofuran
group. “I” stands for deoxyinosine. BHQ2 stands for Black Hole
Quencher 2 and is used as a quencher of the Cy5 fluorophore.

| SEQ | | | |
|---|---|---|---|
| ID | | | |
| NO: | Name | Sequences | Function |
| 73 | CBo12-2P53 | C*T*G*GGaGAATGCTGGGATGAA | aT |
| 74 | pTBoT5S3P | T*T*T*TT CTGGGATGAATG | pT |
| 75 | rTBo-2BsmICy5 | Cy5 *C*T*TCATGAATGCTGGGATGAAG BHQ2 | rT |
| 76 | NBItoBo-2+2P | TG-CTGGGATGAAGTTTGACTCACATTGCTTCA TTT | cT |
| | | TGAAGCAATGTGAGTCAAACTTCTGGACTGTT | |
| 77 | nbitoBo-2+2(rG) | TGCTGGGATGAAGTTTGACTCACATTGCTTCAGC-TTT- | cT |
| | | GCTGAAGCAATgTGAGA | |
| 78 | nbitoBo-2+2AP | TGCTGGGATGAAGTTTGACTCACATTGCTTCA TTT | cT |
| | | TGAAGCAATGTGAGFCAAACTTTTT | |
| 79 | nbitoBo- | TGCTGGGATGAAGTTTGACTCACATTGCTTCA TTT | cT |
| | 2+2UDG (2) | TGAAGCAAUGUGAGTTTTT | |
| 80 | Aagtodna-top | GTAGGTTG TIAATGATGTAGAATGAGT | cT |
| 81 | Aagtodnabot | ACTCATTCTACATCATTTACAACCTAC | cT |
| 82 | dnatoBo-2+2P | TG-CTGGGATGAAGTTTG ACT CAA -ACT ATA CAA | cT |
| | | CCT ACT ACC TCA | |
| 83 | nitoBo- | ATGCCTAATGTCTCA | cT |
| | 2+2BsmAI+9 | TGCTGGGATGAAGTTTGACTCACATTGCTTCA TTT | |
| | | TGAAGCAATGT GAGTCAAAC | |
| 84 | Prec-rna | ugagguaguagguuguauaguu | cT |
| 85 | polyAtoBo-2+2P | TGCTGGGATGAAGTTTG ACT CA | cT |
| | | TTTTTTTTTTTTTTTTTTTTTTTTT | |
| 86 | ligltoBo-2+2 | TGCTGGGATGAAG | cT |
| 87 | lig2P | p CTTGACTCACATTGCTTCATTTTTTGAAGCAATGTG | cT |
| 88 | lig3P | p AGTCAAGCTTCATCTTT | cT |
| 89 | lig2noP | CTTGACTCACATTGCTTCATTTTTTGAAGCAATGTG | cT |
| 90 | lig3noP | AGTCAAGCTTCATCTTT | cT |

Reaction assembly: All reactions were assembled at 4° C. in 200 μL PCR tubes. Templates and enzymes (Table 4) were mixed with the reaction buffer (20 mM Tris HCL pH 7.9, 10 mM (NH₄)₂SO₄, 40 mM KCL, 10 mM MgSO₄, 50 μM each dNTP, 0.1% (w/v) Synperonic F 104, 2 μM netropsin, all purchased from Sigma Aldrich) and BSA (200 μg/mL). Samples were spiked with 10% v/v of various concentration of the targeted enzyme serially diluted in 200 μL PCR tubes with 1× of reaction buffer supplemented with BSA (200 μg/mL). Following an optional preincubation step, sample are incubated at 48° C. in CFX96 touch thermocycler instrument. Detailed experimental conditions are presented in Table 4.

TABLE 4
Experimental conditions for the detection of the different assessed enzymes.

| | | | | | | | | | T4 DNA | | | |
|---|---|---|---|---|---|---|---|---|---|---|---|---|
| | ntBstNBI | RNAseH2 | APE-1 | UDG | AAG | BsmAI | PAP | | ligase | | T4 PNK | |
| aTα | 50 nM | | | | | | | | | | | |
| pTα | 6 nM | 8 nM | 7 nM | 7 nM | 7 nM | 16 nM | 8 | nM | 6 | nM | 7.5 | nM |
| rTα-Cy5 | 30 nM | | | | | | | | | | | |
| cT | nbitoBo- | nbitoBo- | nbitoBo- | nbitoBo- | Aagtolet7atop/ | nbitoBo- | Prec-rna/ | | lig1toBo- | | lig1toBo- | |
| | 2 + 2 | 2 + 2(rG) | 2 + 2AP | 2 + 2UDG(2) | Aagtolet7abot/ | 2 + 2bsmAI + 9 | polyAtoBo- | | 2 + 2/ | | 2 + 2/ | |
| | 8 nM | 200 pM | 50 pM | 100 pM | Let7atoBo- | 50 pM | 2 + 2P | | lig2P/ | | lig2noP/ | |
| | | | | | 2 + 2P | | 10/1 nM | | lig3P | | lig3noP | |
| | | | | | 1/5/1 nM | | | | 33 nM | | 10 nM | |
| | | | | | | | | | each | | each | |
| Nb.Bsml | 300 u/mL | | | | | | | | | | | |
| Vent(exo-) | 70 u/mL | | | | | | | | | | | |
| ttRecJ | 13 nM | | | | | | | | | | | |
| Nt.BstNBI | | 10 u/ml | | | | | | | | | | |
| APE-1 | | | | 2 u/mL | | | | | | | | |
| T4 DNA ligase | | | | | | | | | | | 16 | u/ml |
| Klenow (exo-) | | | | | | | 2.5 | u/mL | | | | |
| ATP | | | | | | | | | 1 | mM | 200 | μM |

| | | | | | | | | | |
|---|---|---|---|---|---|---|---|---|---|
| preincubation | none | none | none | 32° C. 1 h | 31° C. 3 h | none | 31° C. 1 h | 4° C. 12 h | 25° C. 6 h |

Digital Assay: In the case of digital assays, enzymes (mix A) and templates (mix B) were mixed in two separate tubes in reaction buffer 1× to prevent the reaction to start prior to the encapsulation of single enzymes in water-in-oil droplets. For increasing the assay throughput, the inventors resorted to a serial emulsification strategy previously reported (Menezes et al., 2019): mix A, containing varying concentration of the target enzymes, are barcoded with different combinations of three fluorescently Labeled dextrans (dextran Texas Red 70,000 MW, dextran Alexa Fluor 488 3,000 MW and, dextran Cascade Blue 10,000 MW Lysine fixable (ThermoFisher Scientific)). Mix A (Loaded into the pressurized sample changer) and mix B were blend and serially emulsified on chip using a 3-inlet flow-focusing microfluidic PDMS chip. The continuous phase is composed of fluorinated oil (Novec-7500, 3M) containing 1% (w/w) fluorosurfactant (RAN Biotechnologies, MA, USA). The microfluidic mold was prepared with standard soft Lithography techniques using SU-8 photoresist (MicroChem Corp., MA, USA) patterned on a 4-inch silicon wafer and manually aligned using a MJB4 mask aligner (SUSS Microtec). A 10:1 mixture of Sylgard 184 PDMS resin (40 g)/crosslinker (4 g) (Dow Corning, MI, USA) was poured on the mold, degassed under vacuum and baked for 2 hours at 70° C. After curing, the PDMS was peeled off from the wafer and the inlets and outlet holes of 1.5 mm diameter were punched with a biopsy punch (Integra Miltex, PA, USA). The PDMS layer was bound onto a 1 mm thick glass slide (Paul Marienfeld GmbH &t Co. K.G., Germany) immediately after oxygen plasma treatment. Finally, the chip underwent a second baking at 200° C. for 5 hours to make the channels hydrophobic.

Droplet imaging and analysis: Droplets were analyzed by transmission and epifluorescent microscopy. A 70×50×1 mm glass slide (Paul Marienfeld, GmbH &t Co. K.G., Germany) was made hydrophobic by pouring 3 mL Novec 1720 (3M) and baked for 1 minute at 100° C. on a heating plate. 10 μm polystyrene beads (Polysciences, Inc., PA, USA), used as hard spheres spacers, were spotted on the glass slide and left for evaporation at 100° C. The emulsion was deposited on the glass slide and covered with a 22×22 mm coverslip (VWR) treated with Novec 1720. Chambers were sealed with an epoxy glue (Sader) and images were acquired using an epifluorescence microscope Nikon Eclipse Ti equipped with a motorized XY stage (Nikon), a camera Nikon DS-Qi2 and a CoolLed pE-4000 illumination source and an apochromatic 20× (N.A. 0.75, WD 1.0) objective. False-color images were generated with the open source ImageJ software.

Images were analyzed using Mathematica software (Wolfram) using the fluorescent barcodes to sort the different sample populations. The number of negative and positive droplets for each sample allows to compute the target enzyme concentration dictated by Poisson statistics.

Results

Proof of Principle with the Nicking Enzyme Nt.BstNBI

The isothermal signal amplification system used here is based on three encoding deoxyribo-oligonucleotides: the first one is an autocatalytic template (seq. Cbo12-2PS3 SEQ ID NO:73), dual-repeat sequence that catalyzes the exponential replication of the trigger strand using a DNA polymerase (Vent(exo-)) and a nicking enzyme (Nb.BsmI); the second pseudotemplate module (seq. pTBoT5PS3 SEQ ID NO:74) deactivates a fraction of the trigger strands and behaves as a catalytic drain to avoid nonspecific, target-independent amplification caused by leaky reactions; the third reporting module (seq: rTBo-2BsmICy5 SEQ ID NO:75), a profluorescent hairpin-shaped probe, hybridizes to the trigger that, upon polymerization, generates a fluorescence signal. Together, these enzymatic and nucleic components create a bistable molecular circuit that can be used in a variety of ultrasensitive biosensing applications.

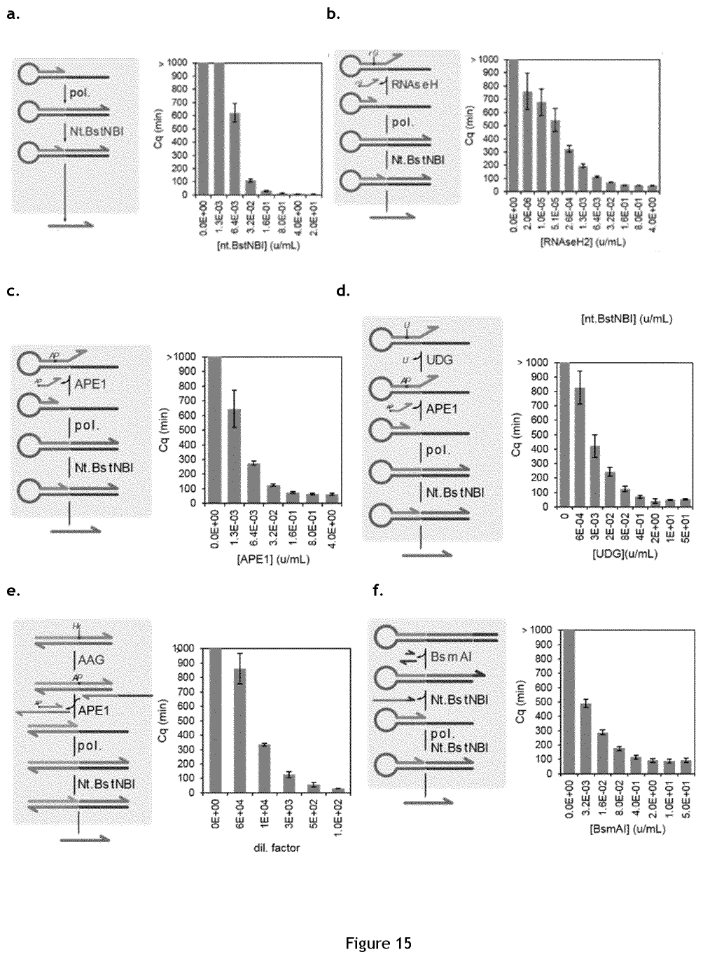

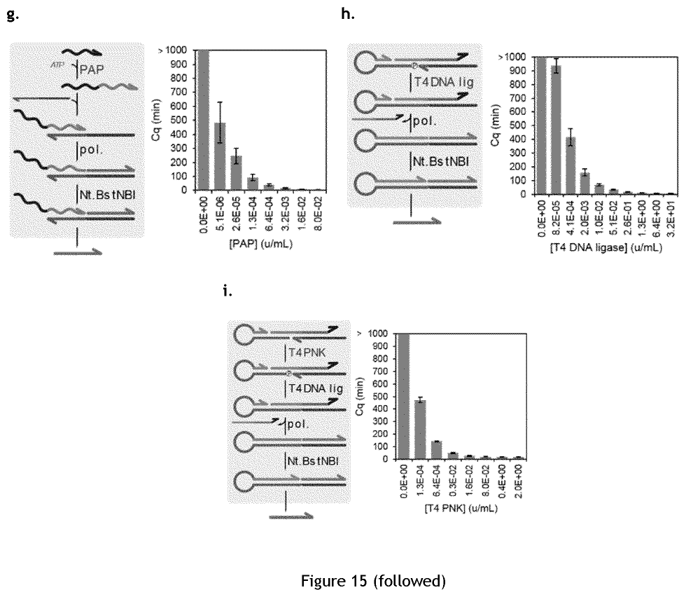

As a proof of principle, the inventors designed a first sensing module (seq: nbitoBo-2+2 SEQ ID NO:76) to connect the activity of nicking enzyme Nt.BstNBI, to the bistable amplification switch (FIG. 15a). The hairpin-shaped template includes a 5′ output site complementary to the trigger strand, just upstream to the nicking recognition and cutting site. The 3′ extremity is self-complementary, priming the extension by the polymerase along the template. In its double-stranded form, the duplex can be nicked by the Nt.BstNBI, releasing the trigger. Catalytic cycles of polymerization/nicking lead to the linear production of the trigger strand that, after exceeding a concentration threshold set by the pseudotemplate, initiates the amplification. The inventors monitored the reaction in real-time in the presence of an increasing concentration of Nt.BstNBI. As expected, the higher the concentration, the faster trigger are produced and therefore, the sooner the amplification. The sensitivity of this approach in bulk is about 5 mu/ml (milliunits per milliliter), which is 3 orders of magnitudes lower than using traditional cleavage assays of profluorescent probes.

Versatility Demonstrated Over 9 Enzymes

Based on these results, the inventors designed a variety of sensing strategies for the ultrasensitive detection of other DNA-related enzymes. A reaction cascade linking the enzymatic activity of interest to the generation of specific trigger strands is designed. The detection of nucleases was based on blocking the trigger production under constitutive presence of Nt.BstNBI. RNAseH (RNAseH2) and AP-endonuclease (APE-1) were detected by introducing in the stem structure of the sensing template a ribonucleotide and an abasic site (AP), respectively (FIGS. 15a and b, seq: nbitoBo-2+2(rG) SEQ ID NO: 77 and nbitoBo-2+2AP SEQ ID NO: 78). In these designs the polymerization of the unprocessed substrate with a protruding 3′ polythimidylate extension was blocked. The processing of these substrates by the corresponding enzyme induces the endonucleolytic cleavage of the stem, restoring the production of triggers by polymerization/nicking cycles. Uracil DNA glycosylase (UDG) was detected by substituting the AP-site by a deoxyribouridine moiety (seq: nbitoBo-2+2UDG(2) SEQ ID NO: 79), adding one more step to the enzymatic cascade (FIG. 15c). Excision of the uracil base by the glycosylase introduces an abasic site that is further incised by APE-1, eventually reactivating the production of triggers.

The inventors tested a different strategy for the detection of another monofunctional DNA N-glycosylase, Alkyl Adenine Glycosylase (AAG, FIG. 15d). An inosine residue (hypoxanthine nucleobase, Hx) is incorporated in a short double-stranded oligonucleotide (seq: Aagtorna-top/Aagtorna-bot, SEQ ID NO:80/SEQ ID NO: 81). Upon excision by AAG, the AP-site is incised by APE-1. The 5′ part of the nicked strand can dissociate and bind to the input part of a second NBI-dependent template, whose output is the trigger strand (seq: dnatoBo-2+2P SEQ ID NO: 82).

The detection of restriction enzymes was achieved by appending the recognition site to the 5′ part of the sensing template (FIG. 15e). In absence of the target enzymes, futile cycles of polymerization/nicking generate unproductive triggers with a 3′ extension (which includes the restriction site). In presence of the target enzyme, the double-stranded restriction site is cleaved, releasing the extension from the sensing template, which in turn produces triggers.

Polymerase with specific activities, such as poly(A) polymerase (PAP), catalyzing the addition of a polyadenine tail to the 3′ extremity of RNA strands can also be detected using this approach (FIG. 15f). Following polyadenylation of an RNA strand (seq: rna SEQ ID NO: 84), the poly(A) tail binds to a poly(T) input site of the sensing template (seq: polyAtoBo-2+2P SEQ ID NO:85), which in turn outputs the trigger.

The inventors also adapted this strategy to the detection of DNA ligase (FIG. 15d). The sensing module are composed of three templates: a hairpin-shaped template (seq: lig2P SEQ ID NO: 87) modified with a 5′ phosphate moiety; a linear template whose 5′ side is complementary to the activator sequence (seq: lig1toBo SEQ ID NO:86); a splint strand which partially hybridizes to both other strands (seq: lig3P SEQ ID NO:88). The resulting triplex includes a nick, which can be sealed by the T4 DNA ligase fueled with ATP. As a consequence, the splint strand is strand-displaced by the DNA polymerase, restoring a functional source of activator strands. The detection of polynucleotide kinase (T4 PNK) was made possible using a non-phosphorylated version of hairpin-shaped template (seq: lig2noP SEQ ID NO: 89). Following its phosphorylation, required for ligation, the T4 DNA ligase can seal the nick, rescuing the production of activator strands.

The inventors performed the detection of each enzyme separately in bulk solution using the cognate design. FIG. 15 shows the amplification time (Cq) extracted from the fluorescence time trace recorded in real-time. For each enzyme the inventors observed an inverted relationship between Cq and the concentrations of the target enzyme, down to the μU to mU/mL concentration range. This demonstrate the specific and sensitive detection of enzyme activities using a versatile DNA amplification mechanism.

Digital Counting of Single Enzymes

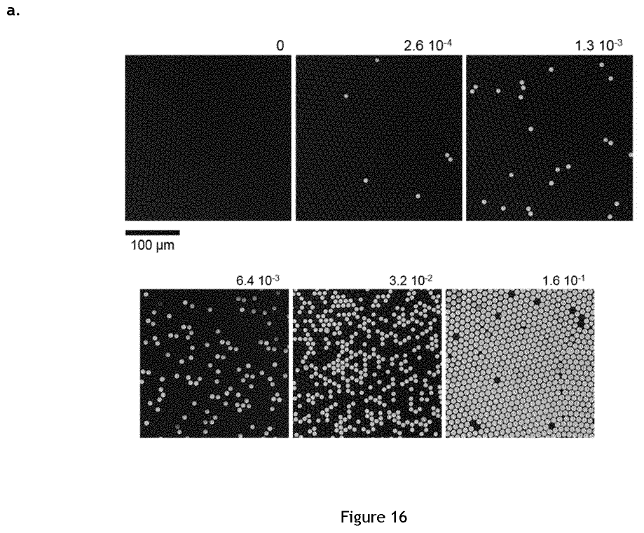

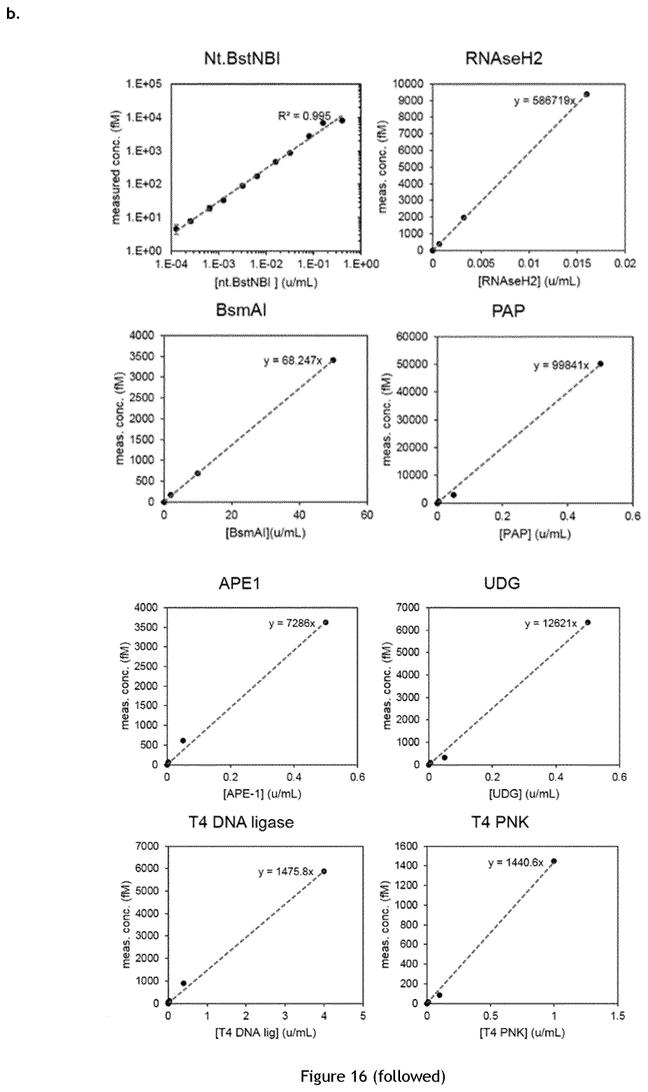

To further demonstrate the sensitivity of this approach, the inventors performed a digital counting of single enzymes isolated in microfluidic droplets. As for bulk assays, the proof of principle was realized with the Nt.BstNBI detection circuit. Two series of samples spiked with different concentrations of NBI were prepared and individually emulsified in picoliter-size droplets (−0.95 pL) using a flow-focusing microfluidic chip. Droplets were incubated at 48° C. for 3 h and analyzed by fluorescence microscopy (FIG. 16a). FIG. 16b (top, left panel) shows the linear correlation between the spiked NBI concentration and the measured concentration computed from the Poisson law (R²>0.99). FIG. 16b presents the results of digital droplet assays for other enzymes including RNAseH2, APE-1, UDG, BsmAI, PAP, T4 DNA ligase and T4 PNK. Similarly, the proportionality between the spike-in concentrations and the measured concentrations proves the success of this approach for digital counting of these enzymes.

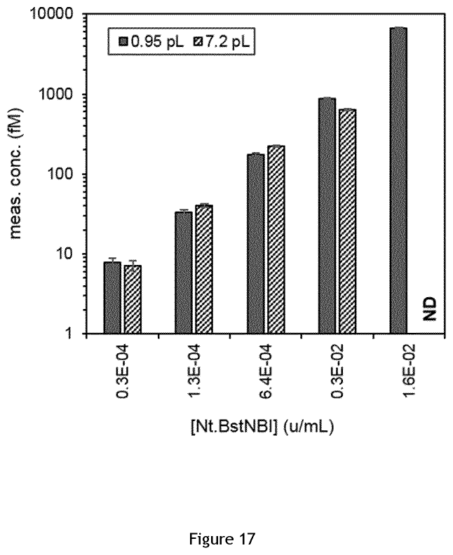

The digital detection of Nt.BstNBI was performed using larger droplets (7.2 pL) (FIG. 17). In comparison with the smaller droplets, very similar concentrations were computed. The proportionality between the spiked concentration and the measured concentration of target, together with the consistent results obtained for different droplet sizes unambiguously demonstrate the direct absolute quantification of active enzymes.

### Example 5: Multiplex Detection of microRNA

Material: HPLC-purified oligonucleotides were purchased from Biomers or Eurofins and resuspended at 100 μM in 1× Tris-EDTA pH 7.5 for long-term storage. Templates were designed according to the protocol described in Example 1 above. Template sequences aT, pT, rT and kT were protected against the 5′->3′ exonuclease activity of ttRecJ by the addition of three 5′ phosphorothioate backbone modifications. Templates aT, pT, cT and kT were blocked for unwanted polymerization by the addition of a 3′ phosphate moiety. aT were designed to hybridize only to the last 10 bases of corresponding input (α or β) in order to favorize the deactivation by pT of signal strands produced by leaky reactions. kT present the same shortened input binding site in order to reduce the competitive binding of signal strands. This prevents the nonspecific activation of kT prior to target-triggered amplification. Table 5 recapitulates all sequences used throughout this assay (SEQ ID NO: 104, 106, 108 and 109 are also cited as SEQ ID NO: 54, 55, 56 and 57 respectively).

TABLE 5
Sequences used in multiplex assay

| SEQ ID NO | Name | Sequence | Function |
|---|---|---|---|
| 101 | α | CATTCAGGATCG | trigger |
| 102 | β | CATTCTGGACTG | trigger |
| 103 | aTα | C*G*A*TCCTGAATG-CGATCCTGAA p | aT |
| 104 | aTβ* | C*A*G*TCCAGAATG-CAGTCCAGAA p | aT |
| 105 | pTα | T*T*T*TTCGATCCTGAATG p | pT |
| 106 | pTβ | T*T*T*TTCAGTCCAGAATG p | pT |
| 107 | rTα | OregonGreen488 *A*T*TCAGAATGCGATCCTGAAT BMNQ535 | rT |
| 108 | rTβ | Atto633 *A*T*TCTGAATGCAGTCCAGAATBHQ2 | rT |
| 109 | let7atoβ | TGCAGTCCAGAA-GTTTGACTCAAACTATACAACCTACTACCTCA | cT |
| 110 | let7etoβ | TGCAGTCCAGAA-GTTTGACTCAAACTATACAACCTCCTACCTCA | cT |
| 111 | mir7toβ | TGCAGTCCAGAA- | cT |
| 112 | mir92atoα | TGCGATCCTGAA-GTTTGACTCAAGCATTGCAACCGATCCCAACC | cT |
| 113 | mir39atoα | TGCGATCCTGAA-GTTTGACTCACAAGCTGATTTACACCC p | cT |
| 91 | αkβ | C*A*T*TCTGGACTGAAAA-CAATGACTCGATCCTGAA p | kT |
| 92 | βkα | C*A*T*TCAGGATCGAAAA-CAATGACTCAGTCCAGAA p | kT |
| 93 | αkβA0O | C*A*T*TCTGGACTG-CAATGACTCGATCCTGAA p | kT |
| 95 | αkβA1 | C*A*T*TCTGGACTGT-CAATGACTCGATCCTGAA p | kT |
| 96 | αkβA2 | C*A*T*TCTGGACTGTT-CAATGACTCGATCCTGAA p | kT |
| 97 | αkβA3 | C*A*T*TCTGGACTGTTT-CAATGACTCGATCCTGAA p | kT |
| 98 | αkβA4 | C*A*T*TCTGGACTGTTTT-CAATGACTCGATCCTGAA p | kT |
| 99 | αkβA5 | C*A*T*TCTGGACTGTTTTT-CAATGACTCGATCCTGAA p | kT |
| 67 | Let7a | ugagguaguagguuguauaguu | microRNA |
| 114 | 1et7e | ugagguaggagguuguauaguu | microRNA |
| 70 | mir39-ce | ucaccggguguaaaucagcuug | microRNA |
| 72 | mir92a | agguugggaucgguugcaaugcu | microRNA |
| 115 | mir7 | uggaagacuagugauuuuguuguu | microRNA |

The nicking enzymes Nb.BsmI and Nt.bstNBI, the restriction enzyme BsmI, the DNA polymerase Vent(exo-), BSA and dNTP were obtained from New England Biolabs (NEB). Thermus thermophilus RecJ exonu-clease was produced in-house following a previously published protocol (Yamagata et al. Nucleic Acids Res. 2001, 29 (22), 4617-4624). Sodium chloride, potassium chloride, magnesium sulfate, ammonium sulfate, Trizma hydrochloride, netropsin, synperonic F104 were purchased from Merck (Sigma-Aldrich).

Reaction mixtures assembly: All reaction mixtures were assembled at 4° C. in 200 μL PCR tubes. Template and enzymes were first mixed with the reaction buffer (20 mM Tris-HCL, pH 8.9, 10 mM (NH4)2SO4, 40 mM KCL, 10 mM MgSO4, 50 μM each dNTP, 0.1% (w/v) synperonic F104, 2 μM netropsin and 200 mg/mL BSA). Optimized template concentrations were as follow: aTα=50 nM, aTβ, 50 nM, pTα=15 nM, pTβ=11 nM, rTα=40 nM, rTβ=40 nM, cT (each)=0.5 nM, αkβ=1 nM and βkα=2.5 nM. Enzyme concentrations were Nb.BsmI=300 u/mL, Nt.BstNBI=10 u/mL, Vent(exo-)=60 u/mL, BsmI=60 u/mL, ttRecJ=23 nM. After homogenization, samples were spiked with microRNA solution, serially diluted in 1× Tris-EDTA buffer using low-binding DNA tips (Eppendorf). Samples (bulk or emulsion) were incubated at 50° C. in a qPCR machine CFX96 touch (Bio-Rad).

Microfluidic droplet generation: A 2-inlet flow focusing device was prepared using standard soft-lithography techniques. Briefly, the microfluidic mold was obtained by coating a 4-inch silicon wafer with SU-8 photoresist (Micro-Chem Corp.) reticulated upon UV exposure. Following careful cleaning of the mold with isopropanol, a 10:1 mixture of Sylgard 184 PDMS resin (40 g)/curing agent (4 g) (Dow Corning) was poured onto the mold, degassed under vacuum and baked for 2 hours at 70° C. The PDMS slab was piled off the mold and inlets and outlets were punched with a 1.5 mm diameter biopsy puncher (Integra Miltex). The PDMS slab was bound on a 1 mm thick glass slide (Paul Marienfeld GmbH &±Co) immediately following oxygen plasma activation. The chip underwent a baking for 5 hours at 200° C. to make the channel hydrophobic. Monodisperse water-in-oil droplets were generated by mixing the aqueous samples and the continuous phase (fluorinated oil Novec 7500, 3M+1% (w/w) fluosurf, Emulseo) on chip using a pressure pump controller MFCS-EZ (Fluigent) and 200 μm inner diameter PTFE tubing (C.I.L.).

Droplet imaging and analysis: Following incubation, emulsions were imaged by microscopy. A monolayer of droplets was sandwiched between two glass slides (1 mm thick bottom slide, Paul Marienfeld GmbH &t Co, 0.17 mm thick top slide, VWR) spaced with 10 μm polystyrene particles (Polysciences, Inc.) to avoid droplet compression. The chamber was sealed with epoxy glue (Sader). Images were acquired on an epifluorescence microscope Eclipse Ti equipped with a motorized XY stage (Nikon), a camera Nikon DS-Qi2, an apochromatic 10× objective (N.A. 0.45, Nikon) and a CoolLed pE-400 illumination source. Composite images were generated with the open source software ImageJ. Droplets were analyzed using the Mathematica software (Wolfram), following the reported procedure described in the above examples. The concentration of microRNA is computed with the formula:

$$[ mir 1 ] = \frac{- ln ( 1 - {F}_{g} )}{{N}_{A} \cdot V} and [ mir 2 ] = \frac{- ln ( 1 - {F}_{r} )}{{N}_{A} \cdot V}$$

where F_{g} and Fᵣ are the fraction of green and red positive droplets respectively, NA is the Avogadro number and V the volume of the droplets.

Results

Killer Templates Counter Switch Cross Activation

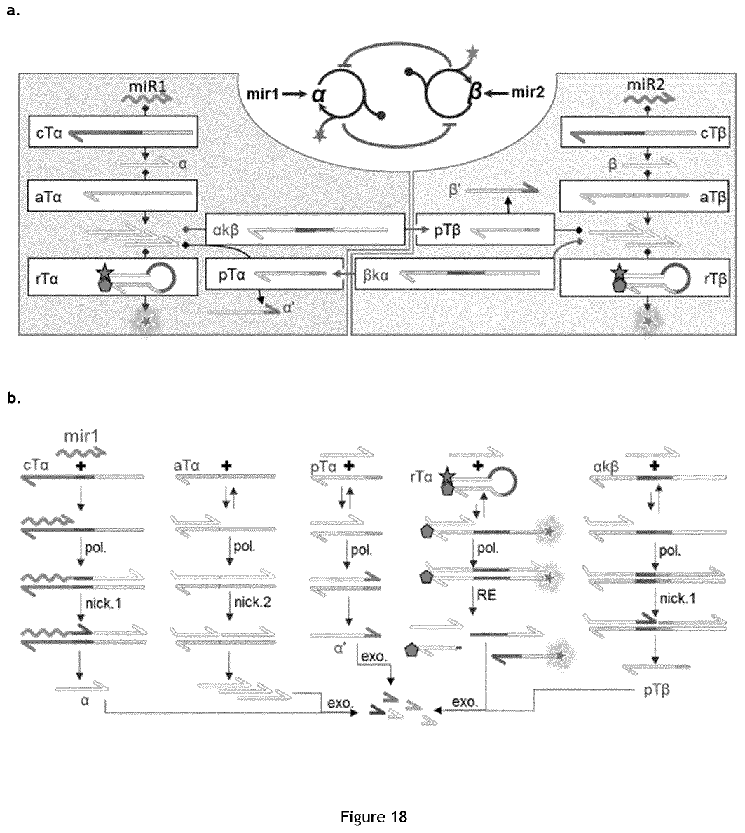

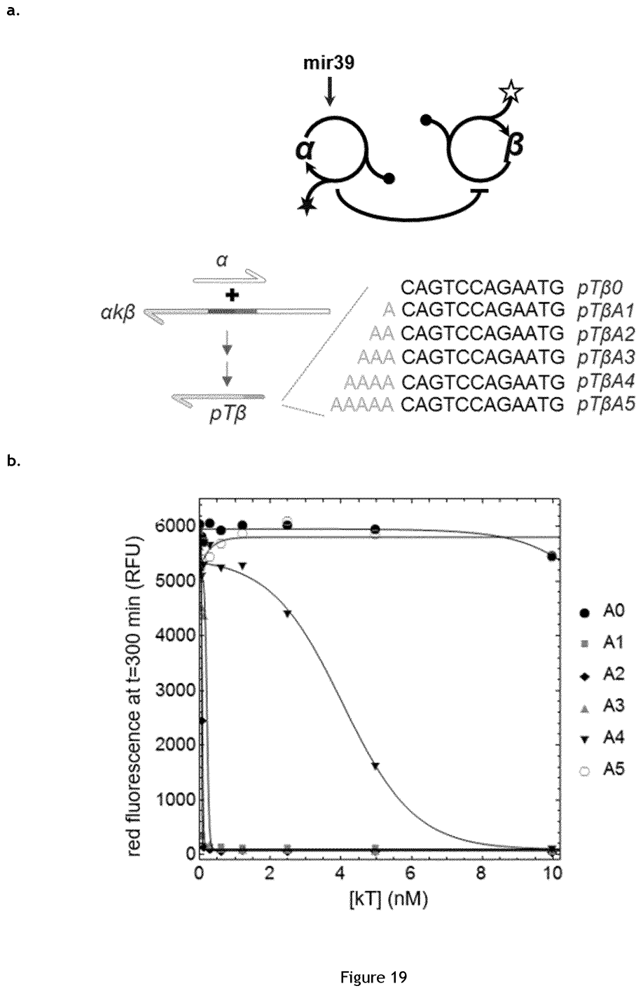

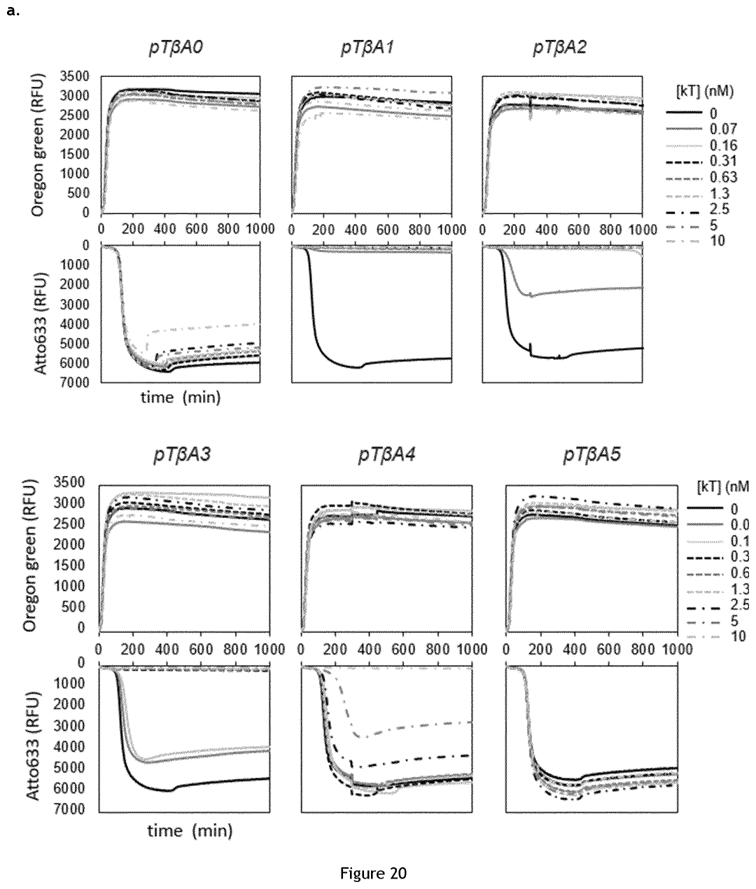

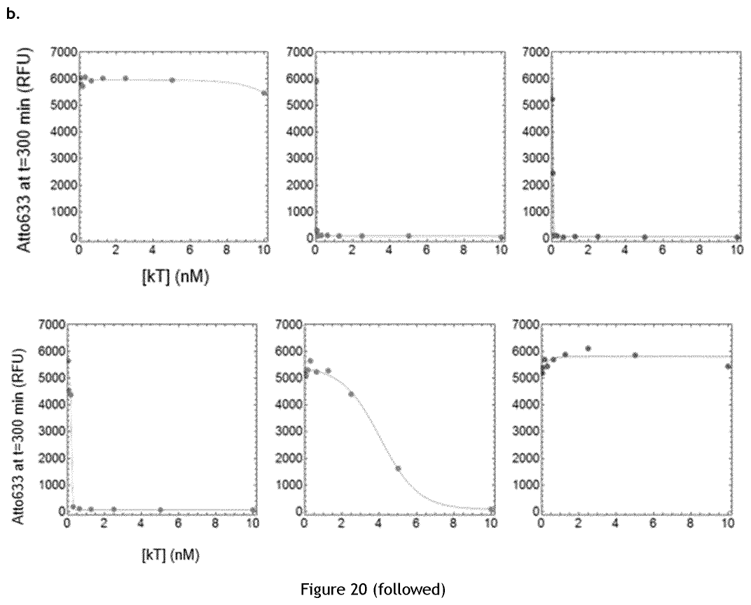

The inventors converted the two parallel bistable switches into a tetrastable biochemical circuit. The rationale is that each of the four alternative states can then be attributed to the four possible chemical “states” associated with the presence/absence of each target (0:0, 0:1, 1:0, and 1:1), allowing appropriate classification in each case. To that goal, the inventors designed cross inhibitory templates (killer templates, kT), which connect the two switches bidirectionally (FIG. 18). Upon activation by their cognate input (α or β), the kT produces a pseudotemplate for the opposite switch, thereby acting as a cross inhibitor of amplification. For the system to admit four states, the inhibitors need to be strong enough to stabilize the state 1:0 and 0:1 (where only one of the two switches is ON), but not too strong to allow the existence of the state 1:1 (where both switches are ON). Accordingly, the inventors evaluated the effect of the length of the endogenous pT—determined by the length of the deactivating 5′ tail on the strength of the killer template. FIG. 19 shows the amplification reaction of a simple β switch in presence of the α switch triggered with 5 nM of cel-mir-39 and an increasing concentration of αkβ producing various pTβ. The system is set in such a way that, in absence of αkβ, the β switch turned on spontaneously in about 100 minutes (FIG. 20).

When increasing the concentration of kT, the inventors observed as expected a growing delay before amplification. Additionally, it is clear that αkβ producing shorter pT, are stronger inhibitors: less than 100 pM of kT αkβA1 (meaning that the resulting pTβ will add only one thymidine nucleo-tide on the 3′ end of the α strand) are required to completely prevent the amplification of the α switch, whereas 100 fold more are needed to observe the same effect with αkβA4 (FIG. 18c). Interestingly, no inhibition was observed in the range of tested concentration for αkβA5. Similarly, the αkβA0 (producing a complementary strand from α with no catalytic extension activity) has no effect on the amplification of the β switch, confirming the catalytic mechanism of the pseudotemplate.

Following these measurements, the inventors opted for kT producing pT with a 4-nucleotide extension, for which the inhibition strength can be easily adjusted by tuning the concentration.

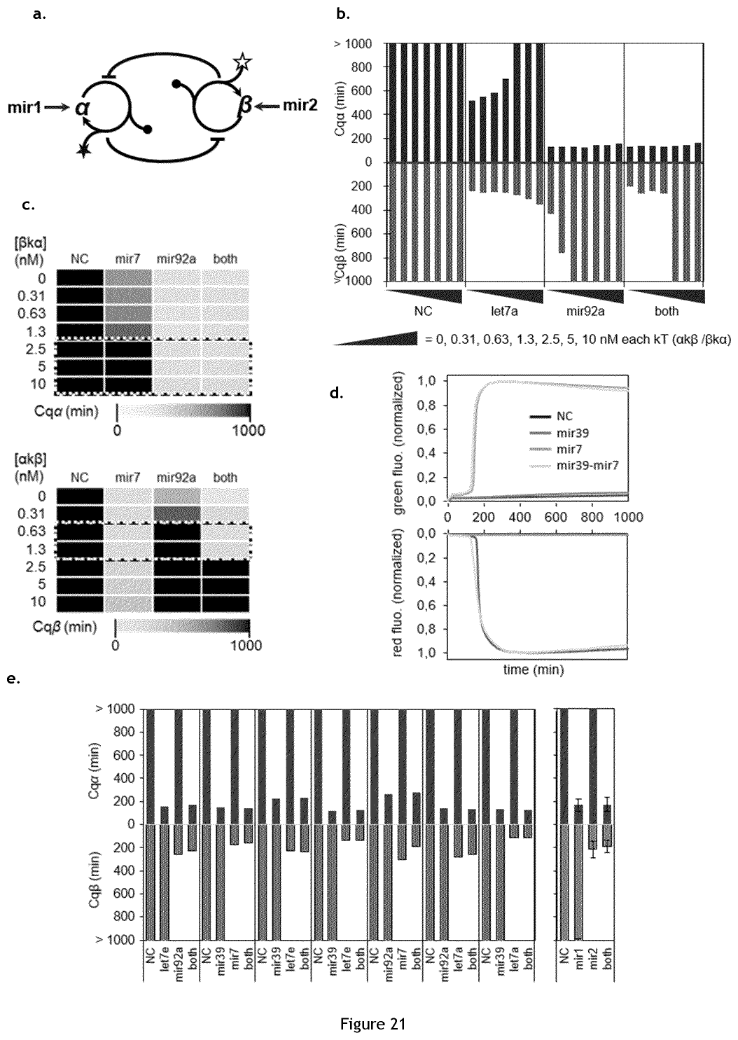

Next, the inventors evaluated the potential of the kT to suppress cross-reactivity between a and β switches, while retaining sensitivity for their cognate target. The two microRNA sensing circuits are spiked with 0 or 10 pM of mir92a (α switch) and let7a (β switch) in presence of various concentrations of both αkβ and βkα (FIG. 21). FIGS. 21b and c show the amplification time of both switches. In these experimental conditions, tetrastability is achieved for 2.5-10 nM of βkα and 0.63-1.3 nM of αkβ: in this concentration range of kT, the absence of target results in the absence of amplification (Cq>1000 min, state 0:0); when only one microRNA target is present, only the corresponding switch amplified a fluorescent signal (Cq−200 min, states 1:0 and 0:1); finally, when both microRNAs are injected, the two switches turned on (state 1:1).

The inventor tested the generalization of this strategy for the detection of other microRNAs. The modular design of this programmable DNA circuit allows in principle the detection of any nucleic acid strand (RNA or DNA) with a known 3′-hydroxyl terminus, by adapting only the converter template's input domain. The rest of the duplex circuit (i.e. both aT, pT, rT and kT) sequences and concentrations remain untouched. For these experiments, the inventors used five microRNAs: has-mir-92a-5p, cel-mir-39, hsa-mir-7-5p, hsa-let-7a-5p and hsa-let-7e-5p (respectively abbreviated mir92a, mir39, mir7, let7a and let7e). FIG. 21e depicts the amplification time (Cq) for seven different duplex experiments in solution for the detection of 0 or 10 pM of microRNA targets. As expected, the system behaves as a tetra-stable biochemical circuit in each case Importantly, the amplification time for each switch is independent of the target sequence (Cqα=178±44 min, Cqβ=149±19 min). Consequently, this confirms that the cross-inhibitory circuit suppresses unwanted cross-reactivity, while enabling programmable target detection.

Duplex Digital Detection of microRNAs.

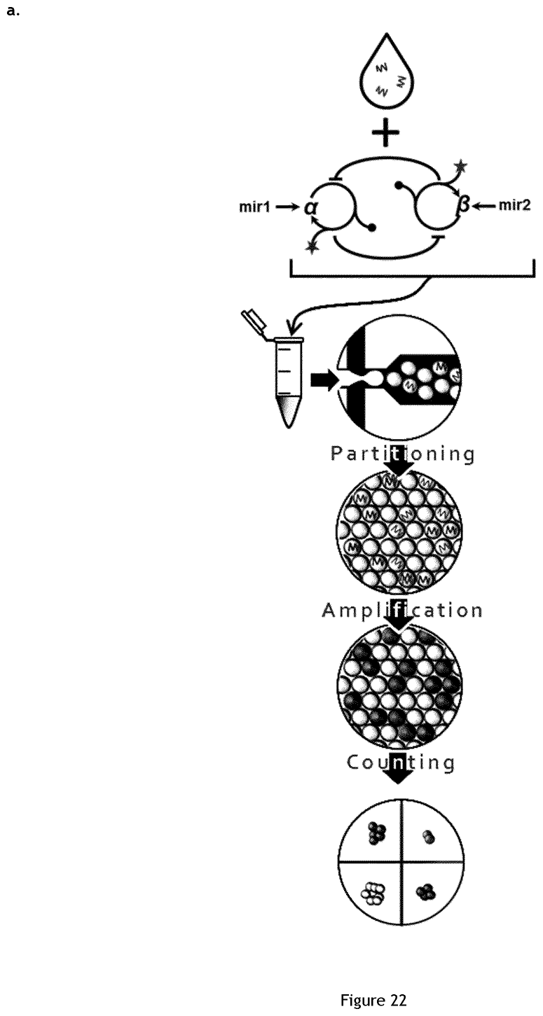

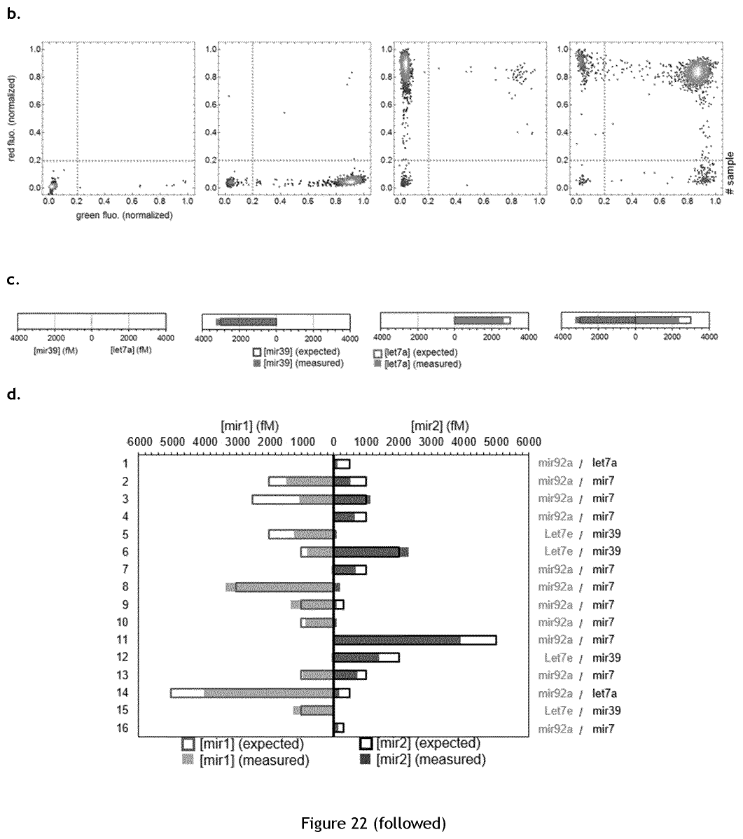

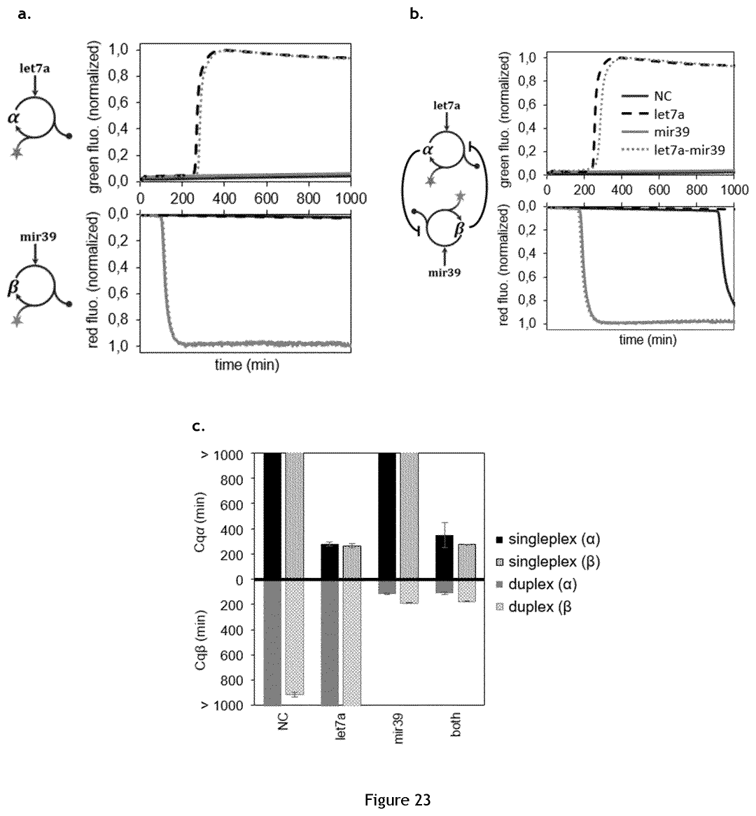

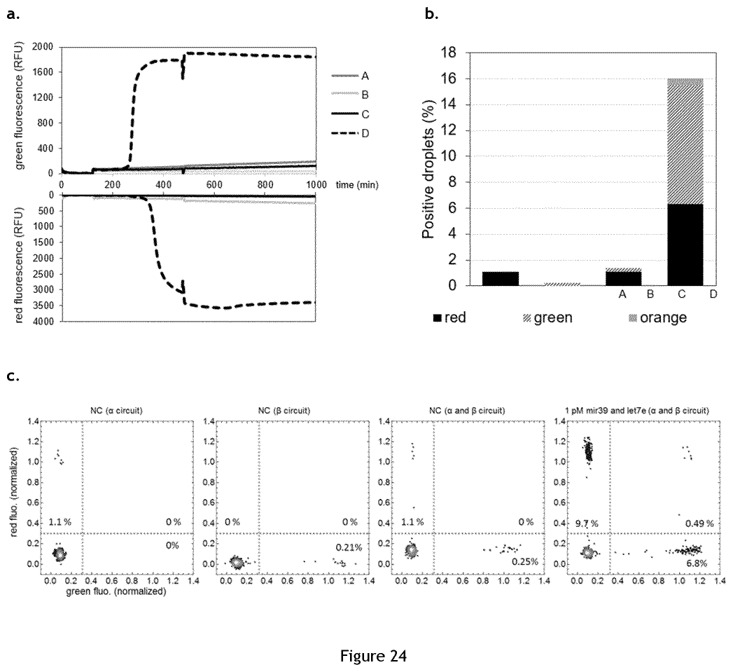

The inventors finally transposed this multiplex assay to a digital readout using droplet microfluidics (FIG. 22. The sample mixture is partitioned into thousands of picolitre-size droplets using a flow-focusing microfluidic device. As a result, target microRNAs are randomly distributed into water-in-oil droplets, with occupancy following a Poissonian distribution. After incubation which allows the droplet fluorescence to turn either green, red or orange depending on their initial content, the droplets are imaged by epifluorescence microscopy. Knowing the droplet size and the fraction of positive droplets in each color, the concentration of the two microRNAs in the original sample was computed. The inventors demonstrated that the tetrastable circuit does not influence the detection (FIG. 23) and the limit of the blank in comparison to singleplex assay (FIG. 24). For better demonstration, the inventors prepared 4 samples spiked with 0 or 3 pM of microRNA mir39 (α switch) and let7a (β switch). Each sample is barcoded with a combination of two fluorescent dextran barcodes and serially emulsified using a homemade sample changer. After incubation, the droplets are imaged by fluorescence microscopy (FIG. 22b-d). While a few false positive events were recorded, an accurate quantification of the two microRNAs within 12%±6% errors (which could be partially due to concentration uncertainties from the serial dilution of the targets) was achieved. To assess the reproducibility of the technique, the inventors repeated this experiment for samples of different compositions (various concentrations of various microRNAs. For the 15 samples, the inventors observed a good correlation between the expected concentration of the spike-in microRNAs and the measured concentration (FIG. 22 d). Finally, the inventors also verified that the fraction of double positive droplets (both green and red droplets) corresponds to the fraction expected from the Poisson distribution of the two targets (Fₒ=F_{g}.Fᵣ, where Fₒ, F_{g} and Fᵣ are the fractions of orange, green and red droplets).

### CONCLUSION

The inventors previously demonstrated that the MP-based isothermal amplification strategy has the potential to completely abolish background amplification. In the present invention, they leverage this feature to convert the analog readout (real-time fluorescence monitoring) to a digital format (end-point compartments analysis). The method of the present invention thus allows the sensitive, specific and quantitative measurement of target biomolecules, particularly of enzymes and nucleic acids, more particularly of microRNA. Based on a one-step procedure, it reduces the sample manipulation and therefore the risk of carry-over contamination. The contamination issue is even more reduced by the fact that the system relies on a signal amplification mechanism rather than target sequence replication.

The versatile DNA-based circuit can be repurposed for any biomolecules of interest, by designing the corresponding conversion template. With respect to microRNA, it should be noted that all other circuits parts are common for all microRNA, thus eliminating the need for primers and probe design and reducing the assay cost.

Moreover, the above results demonstrate that the DNA circuit architecture can be adapted for detecting enzymes, particularly DNA-related enzymes with a wide range of activities (nucleases, DNA N-glycosylases, polymerases, ligases and kinases). The sensitivity of the present method allows for the direct digital counting of individual enzymes isolated in picoliter-size compartment. Said method can also be used for the quantification of active enzymes following the purification process and to determine the effect of physical (temperature) or chemical treatment on the enzymatic activity at the single-enzyme level.

The above examples also demonstrate that the method of the invention may be adapted for multiplex in a detection at more sensitive manner.

### BIBLIOGRAPHICAL REFERENCES

A. Schamberger and T. I. Orban, “3′ IsomiR Species and DNA Contamination Influence Reliable Quantification of MicroRNAs by Stem-Loop Quantitative PCR,” PLOS ONE, vol. 9, no. 8, p. e106315, August 2014.
Aslanzadeh, “Preventing PCR Amplification Carryover Contamination in a Clinical Laboratory,” Ann. Clin. Lab. Sci., vol. 34, no. 4, pp. 389-396, January 2004.
A. Yamagata, R. Masui, Y. Kakuta, S. Kuramitsu, and K. Fukuyama, “Overexpression, purification and characterization of RecJ protein from Thermus thermophilus HB8 and its core domain,” Nucleic Acids Res., vol. 29, no. 22, pp. 4617-4624, November 2001.
Brouzes et al. “Droplet microfluidic technology for single-cell high-throughput screening”, PNAS 2009
Campomenosi, P. et al. A comparison between quantitative PCR and droplet digital PCR technologies for circulating microRNA quantification in human lung cancer. BMC Biotechnol. 16, 60 (2016).
C. A. Raabe, T.-H. Tang, J. Brosius, and T. S. Rozhdestvensky, “Biases in small RNA deep sequencing data,” Nucleic Acids Res., vol. 42, no. 3, pp. 1414-1426, February 2014.
Genot et al, “High-resolution mapping of bifurcations in nonlinear biochemical circuits”, Nat. Chem. 2016.
Huggett et al. BMC Res Notes 2008
H. Jia, Z. Li, C. Liu, and Y. Cheng, “Ultrasensitive Detection of microRNAs by Exponential Isothermal Amplification,” Angew. Chem. Int. Ed., vol. 49, no. 32, pp. 5498-5501, July 2010.
K. L. Opel, D. Chung, and B. R. McCord, “A Study of PCR Inhibition Mechanisms Using Real Time PCR” J. Forensic Sci., vol. 55, no. 1, pp. 25-33, 2010.
K. Montagne, R. Plasson, Y. Sakai, T. Fujii, and Y. Rondelez, “Programming an in vitro DNA oscillator using a molecular networking strategy,” Mol. Syst. Biol., vol. 7, p. 466, February 2011.
K. Montagne, G. Gines, T. Fujii, and Y. Rondelez, “Boosting functionality of synthetic DNA circuits with tailored deactivation,” Nat. Commun., vol. 7, p. 13474, November 2016.
K. Zhang et al., “Digital quantification of miRNA directly in plasma using integrated comprehensive droplet digital detection,” Lab. Chip, September 2015.
Menezes R, Dramé-Maigné A, Taly V, Rondelez Y, Gines G. Streamlined digital bioassays with a 3D printed sample changer. Analyst. 2019 Nov. 21 [cited 2019 Dec. 12]; Available from: https://pubs.rsc.org/en/content/articelanding/2020/an/c9an01744e
L. Cohen, M. R. Hartman, A. Amardey-Wellington, and D. R. Walt, “Digital direct detection of microRNAs using single molecule arrays,” Nucleic Acids Res., vol. 45, no. 14, pp. e137-e137, August 2017.
S. Kaneda, K. Ono, T. Fukuba, T. Nojima, T. Yamamoto, and T. Fujii, “Modification of the glass surface property in PDMS-glass hybrid microfluidic devices,” Anal. Sci. Int. J. Jpn. Soc. Anal. Chem., vol. 28, no. 1, pp. 39-44, 2012.
Y. Zhao, F. Chen, Q. Li, L. Wang, and C. Fan, “Isothermal Amplification of Nucleic Acids,” Chem. Rev., vol. 115, no. 22, pp. 12491-12545, November 2015.

1. A digital method for detecting and/or quantifying at least one target biomolecule in a sample comprising the following steps:
a) mixing said sample with a mixture including a buffer, enzymes, a first oligonucleotide which is an amplification oligonucleotide, a second oligonucleotide which is a leak absorption oligonucleotide, and a third oligonucleotide which is a target-specific conversion oligonucleotide;
b) partitioning the mixture obtained in step a) into several compartments so that a fraction of the compartments does not contain the target biomolecule;
c) converting the target biomolecule into a signal;
d) amplifying the signal, and
e) detecting and/or measuring said signal in each compartment.

2. The digital method of claim 1, wherein the target biomolecule is a nucleic acid or protein.

3. The method according to claim 2, wherein the target biomolecule is a nucleic acid selected from the group consisting of DNA, cDNA, RNA, mRNA, and micro RNA.

4. The method according to claim 1, wherein the enzymes used in step a) are selected from the group consisting of polymerase, nicking enzyme or restriction enzyme, and exonuclease.

5. The method according to claim 1, wherein the first oligonucleotide includes a partial repeat structure containing a nicking enzyme recognition site, and the second oligonucleotide is able to bind, extend, deactivate, and slowly release the products of polymerization along the first oligonucleotide, thereby inducing a threshold effect.

6. The method of claim 1, further comprising adding a fourth oligonucleotide which is a reporting probe.

7. The method of claim 1, further comprising adding a fifth oligonucleotide which is a cross inhibiting oligonucleotide for detecting and/or quantifying two or more biomolecules.

8. The method according to claim 1, wherein the mixture obtained in step a) is partitioned in step b) into droplets.

9. The method according to claim 8, wherein the size of droplet is between 0.001 and 100 pL.

10. The method according of claim 1, wherein said signal is labelled.

11. The method according to claim 1, wherein the step d) of detecting and/or measuring said signal comprises detecting and/or counting the compartments emitted a fluorescence.

12. The method according to claim 11, wherein for measuring the absolute concentration of the target biomolecule in the tested biological sample, the compartments receiving the fluorescent signal and the non-fluorescent compartments are counted and their ratio is calculated.

13. The method according to claim 1, wherein the target biomolecule is used as a biomarker.

14. An in vitro method for diagnosis of a disease selected from the group comprising cancer, neuronal diseases, cardiovascular diseases, inflammatory diseases, autoimmune diseases, diseases due to a viral or bacterial infection, skin diseases, skeletal muscle diseases, dental diseases, and prenatal diseases comprising the use of the method according to claim 1.

15. An in vitro method for agro diagnosis of a disease selected from the group comprising:
diseases caused by biotic stress, or
diseases caused by abiotic stress,
said method comprising the use of the digital method according to claim 1.

16. A kit for detecting and/or quantifying at least one target biomolecule comprising:
a) a mixture of enzymes, selected from the group consisting of polymerase, nicking enzyme or restriction enzymes and exonuclease;
b) a mixture of oligonucleotides comprising a first oligonucleotide which is an amplification oligonucleotide, a second oligonucleotide which is a leak absorption oligonucleotide, and a third oligonucleotide which is a target-specific conversion oligonucleotide and optionally a fourth oligonucleotide which is a reporting probe, and
c) a partitioning agent.

17. The method according to claim 1, wherein the converted signal in step c) is a DNA single strand.

18. The method according to claim 6, wherein the reporting probe is a fluorescent probe.

19. The method according to claim 8, wherein the droplets are water-in-oil emulsion droplets.

20. The in vitro method according to claim 15, wherein:
the diseases caused by biotic stress have an infectious and/or parasitic origin, or
the diseases caused by abiotic stress are caused by nutritional deficiencies and/or unfavorable environment.


SEQUENCE LISTING (115 sequences)

SEQ ID NO: 1 - DNA, 24 bp, Artificial Sequence
  CBe12PS3autocatalytic template
cgatcctgaatgcgatcctgaatg

SEQ ID NO: 2 - DNA, 23 bp, Artificial Sequence
  CBe12-1PS3autocatalytic template
cgatcctgaatgcgatcctgaat

SEQ ID NO: 3 - DNA, 22 bp, Artificial Sequence
  CBe12-2PS3autocatalytic template
cgatcctgaatgcgatcctgaa

SEQ ID NO: 4 - DNA, 17 bp, Artificial Sequence
  ptBe12T5SPpseudotemplate
tttttcgatcctgaatg

SEQ ID NO: 5 - DNA, 23 bp, Artificial Sequence
  CBa12-1PS4autocatalytic template
ctcgtcagaatgctcgtcagaat

SEQ ID NO: 6 - DNA, 16 bp, Artificial Sequence
  ptBa12A4SPpseudotemplate
aaaactcgtcagaatg

SEQ ID NO: 7 - DNA, 17 bp, Artificial Sequence
  ptBa12T5SP pseudotemplate
tttttctcgtcagaatg

SEQ ID NO: 8 - DNA, 16 bp, Artificial Sequence
  ptBa12T4S3Ppseudotemplate
ttttctcgtcagaatg

SEQ ID NO: 9 - DNA, 15 bp, Artificial Sequence
  ptBa12T3S3Ppseudotemplate
tttctcgtcagaatg

SEQ ID NO: 10 - DNA, 14 bp, Artificial Sequence
  ptBa12T2S3Ppseudotemplate
ttctcgtcagaatg

SEQ ID NO: 11 - DNA, 13 bp, Artificial Sequence
  ptBa12T1S3Ppseudotemplate
tctcgtcagaatg

SEQ ID NO: 12 - DNA, 22 bp, Artificial Sequence
  CBa12-2PS4autocatalytic template
ctcgtcagaatgctcgtcagaa

SEQ ID NO: 13 - DNA, 24 bp, Artificial Sequence
  CBa12PS4autocatalytic template
ctcgtcagaatgctcgtcagaatg

SEQ ID NO: 14 - DNA, 23 bp, Artificial Sequence
  CBa12-1PS4autocatalytic template
ctcgtcagaatgctcgtcagaat

SEQ ID NO: 15 - DNA, 21 bp, Artificial Sequence
  CBa12-3PS4autocatalytic template
ctcgtcagaatgctcgtcaga

SEQ ID NO: 16 - DNA, 17 bp, Artificial Sequence
  pTk12T5S4Ppseudotemplate
  misc_feature (13)..(13): n is deoxyuridine
tttttcaatgacncctg

SEQ ID NO: 17 - DNA, 14 bp, Artificial Sequence
  ApTk12A1SUPpseudotemplate
  misc_feature (9)..(9): n is deoxyuridine
acaatgacncctga

SEQ ID NO: 18 - DNA, 15 bp, Artificial Sequence
  ApTk12A2SUPpseudotemplate
  misc_feature (10)..(10): n is deoxyuridine
aacaatgacncctga

SEQ ID NO: 19 - DNA, 16 bp, Artificial Sequence
  ApTk12A3PSpseudotemplate
  misc_feature (11)..(11): n is deoxyuridine
aaacaatgacncctga

SEQ ID NO: 20 - DNA, 17 bp, Artificial Sequence
  ApTk12A4SUPpseudotemplate
  misc_feature (12)..(12): n is deoxyuridine
aaaacaatgacncctga

SEQ ID NO: 21 - DNA, 18 bp, Artificial Sequence
  ApTk12A5SUPpseudotemplate
  misc_feature (13)..(13): n is deoxyuridine
aaaaacaatgacncctga

SEQ ID NO: 22 - DNA, 19 bp, Artificial Sequence
  ApTk12A6SUPpseudotemplate
  misc_feature (14)..(14): n is deoxyuridine
aaaaaacaatgacncctga

SEQ ID NO: 23 - DNA, 22 bp, Artificial Sequence
  Ck12-2PS4 bioteg Autocatalytic template
  misc_feature (8)..(8): n is deoxyuridine
caatgacncctgcaatgactcc

SEQ ID NO: 24 - DNA, 21 bp, Artificial Sequence
  Cba12-2biot3 autocatalytic template
tcgtcagaatgctcgtcagaa

SEQ ID NO: 25 - DNA, 17 bp, Artificial Sequence
  ptBa12A5SP pseudotemplate
aaaaactcgtcagaatg

SEQ ID NO: 26 - DNA, 22 bp, Artificial Sequence
  Cbe12-2S4P autocatalytic template
cgatcctgaatgcgatcctgaa

SEQ ID NO: 27 - DNA, 16 bp, Artificial Sequence
  ApTBe12A3S3P pseudotemplate
aaacgatcctgaatga

SEQ ID NO: 28 - DNA, 22 bp, Artificial Sequence
  CBe12-2noPS3 autocatalytic template
cgatcctgaatgcgatcctgaa

SEQ ID NO: 29 - DNA, 22 bp, Artificial Sequence
  Ck12-2S4noUbioteg autocatalytic template
  misc_feature (8)..(8): n is deoxyuridine
caatgacncctgcaatgactcc

SEQ ID NO: 30 - DNA, 18 bp, Artificial Sequence
  ApTk12A5S3P pseudotemplate
  misc_feature (13)..(13): n is deoxyuridine
aaaaacaatgacncctga

SEQ ID NO: 31 - DNA, 21 bp, Artificial Sequence
  CBe12-2AULP autocatalytic template
cgatcctgaatgcgatcctga

SEQ ID NO: 32 - DNA, 48 bp, Artificial Sequence
  D21tof5TBe12S3P conversion template
cgatcctgaaagcgaagtttgactcatcaacatcagtctgataagcta

SEQ ID NO: 33 - DNA, 21 bp, Artificial Sequence
  CBe12-3noPS3 autocatalytic template
cgatcctgaatgcgatcctga

SEQ ID NO: 34 - DNA, 34 bp, Artificial Sequence
  D21tofBe12S0P conversion template
cgatcctgaatgtcaacatcagtctgataagcta

SEQ ID NO: 35 - DNA, 26 bp, Artificial Sequence
  CBe12-2SPCy355 autocatalytic template
cgatcctgaatgcgatccatcctgaa

SEQ ID NO: 36 - DNA, 22 bp, Artificial Sequence
  CBc12SPBMN35 autocatalytic template
cagtccagaatgcagtccagaa

SEQ ID NO: 37 - DNA, 17 bp, Artificial Sequence
  pTBc12T5SP pseudotemplate
tttttcagtccagaatg

SEQ ID NO: 38 - DNA, 48 bp, Artificial Sequence
  92atoF5TBe12PS0 conversion template
cgatcctgaaagcgaagtttgactcaagcattgcaaccgatcccaacc

SEQ ID NO: 39 - DNA, 48 bp, Artificial Sequence
  Let7atof5TBa12S0P conversion template
ctcgtcagaaagcgaagtttgactcaaactatacaacctactacctca

SEQ ID NO: 40 - DNA, 30 bp, Artificial Sequence
  CBa12-2AULP autocatalytic template
ctcgtcagaatgctcgtcagaaagcgaagc

SEQ ID NO: 41 - DNA, 16 bp, Artificial Sequence
  ApTBa12A3S3P pseudotemplate
aaactcgtcagaatga

SEQ ID NO: 42 - DNA, 28 bp, Artificial Sequence
  RPBe-Cy6 reporting probe
ttttgcattcaattttcgatcctgaatg

SEQ ID NO: 43 - DNA, 23 bp, Artificial Sequence
  CBe12-1S4 bioteg autocatalytic template
cgatcctgaatgcgatcctgaat

SEQ ID NO: 44 - DNA, 24 bp, Artificial Sequence
  Cba12S bioteg autocatalytic template
ctcgtcagaatgctcgtcagaatg

SEQ ID NO: 45 - DNA, 28 bp, Artificial Sequence
  RPBa-Hex reporting probe
ttttgaattctattttctcgtcagaatt

SEQ ID NO: 46 - DNA, 21 bp, Artificial Sequence
  Cba12-3noPS3 autocatalytic template
ctcgtcagaatgctcgtcaga

SEQ ID NO: 47 - DNA, 20 bp, Artificial Sequence
  RPBe-Cy5(2) reporting probe
ttcaggttttcgatcctgaa

SEQ ID NO: 48 - DNA, 21 bp, Artificial Sequence
  RPBe-Cy5(3) reporting probe
attcagaatgcgatcctgaat

SEQ ID NO: 49 - DNA, 18 bp, Artificial Sequence
  ptBa12A6biot pseudo-template
aaaaaactcgtcagaatg

SEQ ID NO: 50 - DNA, 45 bp, Artificial Sequence
  92atof1Be12-3+3 conversion template
atgcgatcctgacgtttgactcaagcattgcaaccgatcccaacc

SEQ ID NO: 51 - DNA, 45 bp, Artificial Sequence
  Let7atof1Ba12-3+3 conversion template
atgctcgtcagacgtttgactcaaactatacaacctactacctca

SEQ ID NO: 52 - DNA, 21 bp, Artificial Sequence
  CBc12-3noPS3 autocatalytic template
cagtccagaatgcagtccaga

SEQ ID NO: 53 - DNA, 20 bp, Artificial Sequence
  RPBc-FAM reporting probe
ttctggttttcagtccagaa

SEQ ID NO: 54 - DNA, 22 bp, Artificial Sequence
  alphatoalpha autocatalytic template
cagtccagaatgcagtccagaa

SEQ ID NO: 55 - DNA, 17 bp, Artificial Sequence
  pTalpha pseudo template
tttttcagtccagaatg

SEQ ID NO: 56 - DNA, 21 bp, Artificial Sequence
  rTalpha reporting template
attctgaatgcagtccagaat

SEQ ID NO: 57 - DNA, 44 bp, Artificial Sequence
  Let7atoalpha conversion template
tgcagtccagaagtttgactcaaactatacaacctactacctca

SEQ ID NO: 58 - DNA, 44 bp, Artificial Sequence
  Let7ctoalpha conversion template
tgcagtccagaagtttgactcaaaccatacaacctactacctca

SEQ ID NO: 59 - DNA, 39 bp, Artificial Sequence
  mir39toalpha conversion template
tgcagtccagaagtttgactcacaagctgatttacaccc

SEQ ID NO: 60 - DNA, 12 bp, Artificial Sequence
  alpha
cattctggactg

SEQ ID NO: 61 - DNA, 22 bp, Artificial Sequence
  alphatoalpha aT
cagtccagaatgcagtccagaa

SEQ ID NO: 62 - DNA, 17 bp, Artificial Sequence
  pTalpha  pT
tttttcagtccagaatg

SEQ ID NO: 63 - DNA, 21 bp, Artificial Sequence
  rTalpha rT
attctgaatgcagtccagaat

SEQ ID NO: 64 - DNA, 44 bp, Artificial Sequence; identical to SEQ ID NO: 57
  Let7atoalpha cT

SEQ ID NO: 65 - DNA, 44 bp, Artificial Sequence; identical to SEQ ID NO: 58
  Let7ctoalpha cT

SEQ ID NO: 66 - DNA, 39 bp, Artificial Sequence; identical to SEQ ID NO: 59
  mir39toalpha cT

SEQ ID NO: 67 - RNA, 22 nt, Artificial Sequence
  Let-7a microRNA
ugagguaguagguuguauaguu

SEQ ID NO: 68 - RNA, 22 nt, Artificial Sequence
  Let-7b microRNA
ugagguaguagguugugugguu

SEQ ID NO: 69 - RNA, 22 nt, Artificial Sequence
  Let-7c microRNA
ugagguaguagguuguaugguu

SEQ ID NO: 70 - RNA, 22 nt, Artificial Sequence
  mir92a microRNA
ucaccggguguaaaucagcuug

SEQ ID NO: 71 - DNA, 55 bp, Artificial Sequence
  Expar-Let7a DNA
aactatacaacctactacctcaaacagactcaaactatacaacctactacctcaa

SEQ ID NO: 72 - RNA, 23 nt, Artificial Sequence
  mir92a synthetic oligoribonucleotide
agguugggaucgguugcaaugcu

SEQ ID NO: 73 - DNA, 22 bp, Artificial Sequence
  CBo12-2PS3
ctgggatgaatgctgggatgaa

SEQ ID NO: 74 - DNA, 17 bp, Artificial Sequence
  pTBoT5S3P  pseudotemplate
tttttctgggatgaatg

SEQ ID NO: 75 - DNA, 22 bp, Artificial Sequence
  rTBo-2BSmICy5 reporter template
cttcatgaatgctgggatgaag

SEQ ID NO: 76 - DNA, 67 bp, Artificial Sequence
  NBItoBo-2+2P
tgctgggatgaagtttgactcacattgcttcattttgaagcaatgtgagtcaaacttctggactgtt

SEQ ID NO: 77 - DNA, 54 bp, Artificial Sequence
  nbitoBo-2+2(rG)
tgctgggatgaagtttgactcacattgcttcagctttgctgaagcaatgtgaga

SEQ ID NO: 78 - DNA, 59 bp, Artificial Sequence
  nbitoBo-2+2AP
tgctgggatgaagtttgactcacattgcttcattttgaagcaatgtgagcaaacttttt

SEQ ID NO: 79 - DNA, 54 bp, Artificial Sequence
  nbitoBo-2+2UDG (2)
  misc_feature (44)..(44): n is deoxyribouridine
  misc_feature (46)..(46): n is deoxyribouridine
tgctgggatgaagtttgactcacattgcttcattttgaagcaangngagttttt

SEQ ID NO: 80 - DNA, 26 bp, Artificial Sequence
  Aagtodna-top
gtaggttgtaatgatgtagaatgagt

SEQ ID NO: 81 - DNA, 27 bp, Artificial Sequence
  Aagtodnabot
actcattctacatcatttacaacctac

SEQ ID NO: 82 - DNA, 44 bp, Artificial Sequence
  dnatoBo-2+2P
tgctgggatgaagtttgactcaaactatacaacctactacctca

SEQ ID NO: 83 - DNA, 70 bp, Artificial Sequence
  nbitoBo-2+2BsmAI+9
atgcctaatgtctcatgctgggatgaagtttgactcacattgcttcattttgaagcaatgtgagtcaaac

SEQ ID NO: 84 - RNA, 22 nt, Artificial Sequence
  Prec-rna
ugagguaguagguuguauaguu

SEQ ID NO: 85 - DNA, 47 bp, Artificial Sequence
  polyAtoBo-2+2P
tgctgggatgaagtttgactcattttttttttttttttttttttttt

SEQ ID NO: 86 - DNA, 13 bp, Artificial Sequence
  lig1toBo-2+2
tgctgggatgaag

SEQ ID NO: 87 - DNA, 36 bp, Artificial Sequence
  lig2P
cttgactcacattgcttcattttttgaagcaatgtg

SEQ ID NO: 88 - DNA, 17 bp, Artificial Sequence
  lig3P
agtcaagcttcatcttt

SEQ ID NO: 89 - DNA, 36 bp, Artificial Sequence; identical to SEQ ID NO: 87
  lig2noP

SEQ ID NO: 90 - DNA, 17 bp, Artificial Sequence
  lig3noP
agtcaagcttcatcttt

SEQ ID NO: 91 - DNA, 34 bp, Artificial Sequence
  akb  (kT)
cattctggactgaaaacaatgactcgatcctgaa

SEQ ID NO: 92 - DNA, 34 bp, Artificial Sequence
  bka (kT)
cattcaggatcgaaaacaatgactcagtccagaa

SEQ ID NO: 93 - DNA, 30 bp, Artificial Sequence
  akbA0 (kT)
cattctggactgcaatgactcgatcctgaa

SEQ ID NO: 94 - DNA, 34 bp, Artificial Sequence; identical to SEQ ID NO: 92
  bkaT4 (kT)

SEQ ID NO: 95 - DNA, 31 bp, Artificial Sequence
  akbA1 (kT)
cattctggactgtcaatgactcgatcctgaa

SEQ ID NO: 96 - DNA, 32 bp, Artificial Sequence
  akbA2  (kT)
cattctggactgttcaatgactcgatcctgaa

SEQ ID NO: 97 - DNA, 33 bp, Artificial Sequence
  akbA3 (kT)
cattctggactgtttcaatgactcgatcctgaa

SEQ ID NO: 98 - DNA, 34 bp, Artificial Sequence
  akbA4 (kT)
cattctggactgttttcaatgactcgatcctgaa

SEQ ID NO: 99 - DNA, 35 bp, Artificial Sequence
  akbA5 (kT)
cattctggactgtttttcaatgactcgatcctgaa

SEQ ID NO: 100 - DNA, 34 bp, Artificial Sequence; identical to SEQ ID NO: 91
  akbT4 (kT)

SEQ ID NO: 101 - DNA, 12 bp, Artificial Sequence
  a trigger
cattcaggatcg

SEQ ID NO: 102 - DNA, 12 bp, Artificial Sequence
  b trigger
cattctggactg

SEQ ID NO: 103 - DNA, 22 bp, Artificial Sequence
  aTa (aT)
cgatcctgaatgcgatcctgaa

SEQ ID NO: 104 - DNA, 22 bp, Artificial Sequence
  aTb  (aT)
cagtccagaatgcagtccagaa

SEQ ID NO: 105 - DNA, 17 bp, Artificial Sequence
  pTa (pT)
tttttcgatcctgaatg

SEQ ID NO: 106 - DNA, 17 bp, Artificial Sequence
  pTb  (pT)
tttttcagtccagaatg

SEQ ID NO: 107 - DNA, 21 bp, Artificial Sequence
  rTa (rT)
attcagaatgcgatcctgaat

SEQ ID NO: 108 - DNA, 21 bp, Artificial Sequence
  rTb  (rT)
attctgaatgcagtccagaat

SEQ ID NO: 109 - DNA, 44 bp, Artificial Sequence; identical to SEQ ID NO: 57
  let7atob  (cT)

SEQ ID NO: 110 - DNA, 44 bp, Artificial Sequence
  let7etob  (cT)
tgcagtccagaagtttgactcaaactatacaacctcctacctca

SEQ ID NO: 111 - DNA, 46 bp, Artificial Sequence
  mir7tob  (cT)
tgcagtccagaagtttgactcaaacaacaaaatcactagtcttcca

SEQ ID NO: 112 - DNA, 44 bp, Artificial Sequence
  mir92ato (cT)
tgcgatcctgaagtttgactcaagcattgcaaccgatcccaacc

SEQ ID NO: 113 - DNA, 39 bp, Artificial Sequence
  mir39ato (cT)
tgcgatcctgaagtttgactcacaagctgatttacaccc

SEQ ID NO: 114 - RNA, 22 nt, Artificial Sequence
  Let7e (microRNA)
ugagguaggagguuguauaguu

SEQ ID NO: 115 - RNA, 24 nt, Artificial Sequence
  mir7 (microRNA)
uggaagacuagugauuuuguuguu